(19) United States
(12) Patent Application Publication
(10) Pub. No.: US 2021/0315978 A1
(43) Pub. Date: Oct. 14, 2021

(54) FORMULATIONS FOR IMPROVED STABILITY OF RECOMBINANT HUMAN PARATHYROID HORMONE

(71) Applicant: SHIRE-NPS PHARMACEUTICALS, INC., Lexington, MA (US)

(72) Inventors: Nitin DIXIT, Lexington, MA (US); Vinh NGUYEN, Lexington, MA (US); Pierre SOUILLAC, Lexington, MA (US); Sujii BASU, Lexington, MA (US)

(73) Assignee: SHIRE-NPS PHARMACEUTICALS, INC., Lexington, MA (US)

(21) Appl. No.: 17/264,720

(22) PCT Filed: Jul. 12, 2019
(86) PCT No.: PCT/US19/41609

§ 371 Date: Jan. 29, 2021

Related U.S. Application Data
(60) Provisional application No. 62/711,767, filed on Jul. 30, 2018.

(51) Int. Cl. A61K 38/29; A61K 9/00; A61K 47/34; A61K 47/10; A61K 9/19; A61K 47/26; A61K 47/22; A61K 47/12; A61K 47/20; A61K 47/02

(52) U.S. Cl. CPC A61K 38/29; A61K 9/0019; A61K 47/34; A61K 47/10; A61K 47/02; A61K 47/26; A61K 47/22; A61K 47/12; A61K 47/20; A61K 9/19

(57) ABSTRACT

The invention relates to pharmaceutical compositions and dosage forms comprising full-length recombinant human parathyroid hormone (rhPTH(1-84)). The invention further relates to new and/or improved PTH compositions having improved in-use stability that are resistant to protein degradation in response to physical and chemical stresses.

### FIELD OF INVENTION

The present invention relates to new and improved pharmaceutical compositions and dosage forms comprising recombinant human parathyroid hormone (rhPTH(1-84)) having improved in-use stability.

### BACKGROUND OF THE INVENTION

Parathyroid hormone (PTH) is a secreted, 84 amino acid product of the mammalian parathyroid gland that controls serum calcium levels through its action on various tissues, including bone. Studies in humans with certain forms of PTH have demonstrated an anabolic effect on bone, and have prompted significant interest in its use for the treatment of osteoporosis and related bone disorders.

Unlike other proteins that have been successfully formulated, PTH is particularly sensitive to various forms of degradation. Unlike other proteins that have been formulated successfully, PTH is particularly sensitive to oxidation, and further requires that its N-terminal sequence remain intact in order to preserve bioactivity. For example, oxidation can occur at methionine residues at positions 8 and 18, giving rise to the oxidized PTH species ox-M(8)-PTH and ox-M(18)-PTH, while deamidation can occur at asparagine in position 16, giving rise to d16-PTH. The polypeptide chain becomes truncated by breakage of peptide bonds, both at the N- and C-terminals. Furthermore, PTH may also be adsorbed to surfaces, form unspecific aggregates and/or precipitate, thus reducing the available concentration of the drug. All these degradation reactions, and combinations thereof, leads to partial or complete loss of PTH bioactivity.

Commercial exploitation of parathyroid hormone requires a formulation that is acceptable in terms of storage and in-use stability and ease of preparation and reconstitution. Because it is a protein and thus far more labile than the traditional small molecular weight drugs, the formulation of parathyroid hormone presents challenges not commonly encountered by the pharmaceutical industry.

A full-length rhPTH(1-84) has recently been approved as a safe and effective treatment for hypoparathyroidism (sold by Shire Pharmaceuticals under the brand name) NATPARA®/NATPAR®). It is the first specific hormone replacement for hypoparathyroidism, and is a once-daily subcutaneous injectable, to be taken as an adjunct to calcium and vitamin D. NATPARA® is currently supplied as a multiple dose, dual-chamber glass cartridge containing a sterile lyophilized powder and diluent in various dose strengths. The sterile lyophilized powder contains either 0.40 mg, or 0.80 mg, or 1.21 mg, or 1.61 mg of parathyroid hormone depending on dose strength and 4.5 mg sodium chloride, 30 mg mannitol, and 1.26 mg citric acid monohydrate. The weight of the sterile diluent is 1.13 g and the diluent contains a 3.2 mg/mL aqueous solution of m-cresol. Upon reconstitution, each dose consists of a solution of rhPTH(1-84) at a pH between 5 and 6.

The disposable NATPARA® medication cartridge is designed for use with a reusable mixing device for product reconstitution and a reusable Q-Cliq pen for drug delivery. The Q-Cliq pen delivers a fixed volumetric dose of 71.4 μL. Using the Q-Cliq pen, each NATPARA® dual-chamber cartridge delivers 14 doses of NATPARA.®

It has been observed that under certain circumstances reconstituted NATPARA® solutions may form protein particulates during the in-use period. Thus, greater robustness of the NATPARA® formulation against physical and chemical stresses encountered during the normal processing conditions, product shelf- and in-use life is desired.

Thus, there exists a need for improved PTH formulations comprising full-length rhPTH(1-84), particularly formulations that prevent physical and chemical degradation of PTH, have improved in-use stability, and ease of preparation, reconstitution, and use.

### SUMMARY OF THE INVENTION

Various non-limiting aspects and embodiments of the invention are described below.

In one aspect, a stable, liquid pharmaceutical formulation comprising recombinant human parathyroid hormone (rhPTH(1-84)) is provided. This formulation is designed for use directly as a liquid for injection, without the step of reconstituting a powder. In one embodiment, the pharmaceutical formulation comprises:
(a) a therapeutically effective amount of recombinant human parathyroid hormone (rhPTH(1-84));
(b) a surfactant;
(c) a tonicity agent;
(d) an antioxidant;
(e) a preservative;
(f) a pharmaceutically acceptable buffer, and
(g) water,
   wherein said pharmaceutical formulation is formulated as a liquid for injection, and wherein the formulation is physically and chemically stable and remains clear, colorless, and free of visible particles for at least 48 hours.

In one embodiment, the pharmaceutical formulation remains clear, colorless, and free of visible particles for at least 72 hours. In one embodiment, the pharmaceutical formulation remains clear, colorless, and free of visible particles for at least 96 hours. In one embodiment, the pharmaceutical formulation remains clear, colorless, and free of visible particles for at least 7 days. In one embodiment, the pharmaceutical formulation remains clear, colorless, and free of visible particles for at least 14 days. In one embodiment, the pharmaceutical formulation remains clear, colorless, and free of visible particles for at least 21 days.

In one embodiment, the surfactant is a poloxamer. In one embodiment, the surfactant is Poloxamer-188. In one embodiment, the surfactant is Poloxamer-188 present at about 0.03 to about 3% w/v of the formulation.

In one embodiment, the tonicity agent is selected from sodium chloride, sucrose, and glycerol, or combinations thereof. In one embodiment, the tonicity agent is sodium chloride present at about 0.2 to about 20% w/v of the formulation. In one embodiment, the tonicity agent is sucrose present at about 0.2 to about 20% w/v of the formulation. In one embodiment, the tonicity agent is glycerol present at about 0.2 to about 20% w/v of the formulation.

In one embodiment, the preservative is m-cresol present at about 0.03 to about 3% w/v of the formulation. In one embodiment, the preservative is m-cresol present at about 0.3% w/v of the formulation.

In one embodiment, the pharmaceutically acceptable buffer is acetate buffer, phosphate buffer, L-Histidine buffer, or succinate buffer. In one embodiment, the pharmaceutically acceptable buffer is present at a concentration of about 5 mM to about 50 mM, or about 20 mM.

In one embodiment, the antioxidant is methionine and it is present at a concentration of about 0.015% to about 1.50% w/v of the formulation. In one embodiment, the antioxidant is methionine present at about 0.15% w/v or 10 mM.

In one embodiment, the pharmaceutical formulation has a pH of about 3.8 to about 6.2, or about 5.5.

The pharmaceutical formulation of claim 1, wherein the formulation is in a unit-dose vial, a multi-dose vial, a cartridge, a pre-filled syringe, an auto-injector, or an injection pen.

In one embodiment, the pharmaceutical formulation comprises:
(a) about 0.2 to about 2.0 mg/mL recombinant human parathyroid hormone (rhPTH(1-84));
(b) about 0.03% to about 3.0% w/v surfactant;
(c) about 0.2% to about 20% w/v tonicity agent;
(d) about 0.015% to about 1.50% w/v antioxidant;
(e) about 0.03% to about 3% preservative;
(f) about 5 mM to about 50 mM pharmaceutically acceptable buffer, and
(g) water,
   wherein said pharmaceutical formulation is formulated as a liquid for injection, and wherein the formulation is physically and chemically stable and remains clear, colorless, and free of visible particles for at least 48 hours.

In one embodiment, the pharmaceutical formulation remains clear, colorless, and free of visible particles for at least 72 hours. In one embodiment, the pharmaceutical formulation remains clear, colorless, and free of visible particles for at least 96 hours. In one embodiment, the pharmaceutical formulation remains clear, colorless, and free of visible particles for at least 7 days. In one embodiment, the pharmaceutical formulation remains clear, colorless, and free of visible particles for at least 14 days. In one embodiment, the pharmaceutical formulation remains clear, colorless, and free of visible particles for at least 21 days.

In another aspect, a pharmaceutical formulation comprising recombinant human parathyroid hormone (rhPTH(1-84)) is provided as a lyophilized powder to be reconstituted prior to injection. In one embodiment, the pharmaceutical formulation comprises:
(a) a therapeutically effective amount of recombinant human parathyroid hormone (rhPTH(1-84));
(b) a bulking agent;
(c) a cryoprotectant, and
(d) a pharmaceutically acceptable buffer,
   wherein said pharmaceutical formulation is formulated as a lyophilized powder to be reconstituted prior to injection, and wherein the formulation is physically and chemically stable and remains clear, colorless, and free of visible particles for at least 48 hours after reconstitution.

In one embodiment, the pharmaceutical formulation remains clear, colorless, and free of visible particles for at least 72 hours. In one embodiment, the pharmaceutical formulation remains clear, colorless, and free of visible particles for at least 96 hours. In one embodiment, the pharmaceutical formulation remains clear, colorless, and free of visible particles for at least 7 days. In one embodiment, the pharmaceutical formulation remains clear, colorless, and free of visible particles for at least 14 days. In one embodiment, the pharmaceutical formulation remains clear, colorless, and free of visible particles for at least 21 days.

In one embodiment, the bulking agent is mannitol. In one embodiment, the bulking agent is mannitol present at about 0.3% to about 30% w/v of the formulation.

In one embodiment, the cryoprotectant is sucrose. In one embodiment, the cryoprotectant is sucrose present at about 0.2 to about 20% w/v of the formulation.

In one embodiment, the pharmaceutically acceptable buffer is phosphate buffer, L-Histidine buffer, or succinate buffer. In one embodiment, the pharmaceutically acceptable buffer is present at a concentration of about 5 mM to about 50 mM, or about 20 mM. In one embodiment, the pharmaceutically acceptable buffer is L-Histidine buffer. In one embodiment, the pharmaceutically acceptable buffer is succinate buffer.

In one embodiment, the pharmaceutical formulation further comprises an antioxidant. In one embodiment, the antioxidant is methionine. In one embodiment, the antioxidant is methionine and it is present at a concentration of about 0.015% to about 1.50% w/v of the formulation. In one embodiment, the antioxidant is methionine present at about 0.15% w/v or 10 mM.

In one embodiment, the pharmaceutical formulation further comprises a surfactant. In one embodiment, the surfactant is a poloxamer. In one embodiment, the surfactant is Poloxamer-188. In one embodiment, the surfactant is Poloxamer-188 present at about 0.03 to about 3% w/v of the formulation.

In one embodiment, the pharmaceutical formulation has a pH of about 3.8 to about 6.2, or about 4.3, or about 5.5.

In one embodiment, the pharmaceutical formulation comprises:
(a) about 0.02 to about 2.0 mg/mL recombinant human parathyroid hormone (rhPTH(1-84));
(b) about 0.3% to about 30% w/v bulking agent;
(c) about 0.2% to about 20% w/v cryoprotectant, and
(d) about 5 mM to about 50 mM pharmaceutically acceptable buffer,
   wherein said pharmaceutical formulation is formulated as a lyophilized powder to be reconstituted prior to injection, and wherein the formulation is physically and chemically stable and remains clear, colorless, and free of visible particles for at least 48 hours after reconstitution.

In one embodiment, the pharmaceutical formulation remains clear, colorless, and free of visible particles for at least 72 hours. In one embodiment, the pharmaceutical formulation remains clear, colorless, and free of visible particles for at least 96 hours. In one embodiment, the pharmaceutical formulation remains clear, colorless, and free of visible particles for at least 7 days. In one embodiment, the pharmaceutical formulation remains clear, colorless, and free of visible particles for at least 14 days. In one embodiment, the pharmaceutical formulation remains clear, colorless, and free of visible particles for at least 21 days.

These and other aspects of the present invention will become apparent to those skilled in the art after a reading of the following detailed description of the invention, including the appended claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a comparison of opalescence of reference suspensions (RS).

FIG. 2 shows the appearance for rhPTH formulated in different buffers upon agitation in 2R glass vials at ambient conditions (220 rotations per minute (rpm), orbital shaking).

FIGS. 3A-3C show RP-HPLC data for the main peak of rhPTH for the pH screen samples stored at 40, 25, and 5° C., respectively, for up to 6 months.

FIGS. 4A-4C show RP-HPLC data for the oxidized Met8 rhPTH impurity for the pH screen samples stored at 40, 25, and 5° C., respectively, for up to 6 months.

FIGS. 5A-5C show RP-HPLC data for the oxidized Met18 rhPTH impurity for the pH screen samples stored at 40, 25, and 5° C., respectively, for up to 6 months.

FIGS. 6A-6C show RP-HPLC data for the IsoAsp33 rhPTH for the pH screen samples stored at 40, 25, and 5° C., respectively, for up to 6 months.

FIGS. 7A-7C show RP-HPLC data for the rhPTH((1-30)+(1-33)) impurities for the pH screen samples stored at 40, 25, and 5° C., respectively, for up to 6 months.

FIGS. 8A-8C show RP-HPLC data for the rhPTH(1-45) fragment impurity for the pH screen samples stored at 40, 25, and 5° C., respectively, for up to 6 months.

FIGS. 9A-9C show RP-HPLC data for the main peak of rhPTH for samples formulated in pH 5.5 acetate buffer containing 50 mM NaCl with different excipients and stored at 40, 25, and 5° C., respectively.

FIGS. 10A-10C show RP-HPLC data for oxidized Met8 rhPTH impurity for samples formulated in pH 5.5 acetate buffer containing 50 mM NaCl with different excipients and stored at 40, 25, and 5° C., respectively.

FIGS. 11A-11C show RP-HPLC data for oxidized Met18 rhPTH impurity for samples formulated in pH 5.5 acetate buffer containing 50 mM NaCl with different excipients and stored at 40, 25, and 5° C., respectively.

FIGS. 12A-12C show RP-HPLC data for IsoAsp33 rhPTH impurity for samples formulated in pH 5.5 acetate buffer containing 50 mM NaCl with different excipients and stored at 40, 25, and 5° C., respectively.

FIG. 13 shows the appearance of the lyophilized cakes for rhPTH formulations according to various embodiments of the disclosure.

### DETAILED DESCRIPTION

Detailed embodiments of the present invention are disclosed herein; however, it is to be understood that the disclosed embodiments are merely illustrative of the invention that may be embodied in various forms. In addition, each of the examples given in connection with the various embodiments of the invention is intended to be illustrative, and not restrictive. Therefore, specific structural and functional details disclosed herein are not to be interpreted as limiting, but merely as a representative basis for teaching one skilled in the art to variously employ the present invention.

Unless defined otherwise, all technical and scientific terms used herein have the same meaning as commonly understood by one of ordinary skill in the art to which this invention belongs.

As used in this specification and the appended claims, the singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

As used in this application, the terms “about” and “approximately” are used as equivalents. Any numerals used in this application with or without about/approximately are meant to cover any normal fluctuations appreciated by one of ordinary skill in the relevant art. As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used herein, the terms “carrier” and “diluent” refers to a pharmaceutically acceptable (e.g., safe and non-toxic for administration to a human) carrier or diluting substance useful for the preparation of a pharmaceutical formulation. Exemplary diluents include sterile water, bacteriostatic water for injection (BWFI), a pH buffered solution (e.g. phosphate-buffered saline), sterile saline solution, Ringer's solution or dextrose solution.

The terms “treat” or “treatment” of a state, disorder or condition include: (1) preventing, delaying, or reducing the incidence and/or likelihood of the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

A “subject” or “patient” or “individual” or “animal”, as used herein, refers to humans, veterinary animals (e.g., cats, dogs, cows, horses, sheep, pigs, etc.) and experimental animal models of diseases (e.g., mice, rats). In a preferred embodiment, the subject is a human.

As used herein the term “effective” applied to dose or amount refers to that quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

The phrase “pharmaceutically acceptable”, as used in connection with compositions of the invention, refers to molecular entities and other ingredients of such compositions that are physiologically tolerable and do not typically produce untoward reactions when administered to a mammal (e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans.

The compositions according to the invention possess improved in-use stability of rhPTH(1-84) as compared to commercially available rhPTH(1-84) formulations. The term “in-use,” as used herein, refers to the period of time during which a multidose formulation can be used while retaining quality within an accepted specification, once the multidose container is opened. “In-use stability,” therefore, refers to the stability of a multidose formulation during the in-use period. In some embodiments of the invention, the in-use period is 7 days. In some embodiments of the invention, the in-use period is 14 days. In some embodiments of the invention, the in-use period is 21 days. In some embodiments of the invention, the in-use period is one month.

rhPTH(1-84)

The compositions disclosed herein incorporate as the active ingredient the full length, 84 amino acid form of human parathyroid hormone, obtained either recombinantly, by peptide synthesis or by extraction from human fluid. In this specification, the recombinant human form of PTH is abbreviated rhPTH(1-84), which has the amino acid sequence reported by Kimura et al, Biochem Biophys Res Comm, 114 (2):493.

As an alternative to the full length human form of PTH, the compositions of the invention may incorporate those homologues, fragments, or variants of human PTH that have human PTH activity as determined in the ovarectomized rat model of osteoporosis reported by Kimmel et al, Endocrinology, 1993, 32(4):1577 and incorporated herein by reference.

In one aspect, the parathyroid hormone compositions of the present invention are provided in a single-unit or multi-unit liquid dosage form, as an aqueous hormone solution for injection that does not require any reconstitution, dilution, or mixing.

In one aspect, the parathyroid hormone compositions of the present invention are provided in a lyophilized powder dosage form containing not more than 3% water by weight, that results from the freeze-drying of a sterile, aqueous hormone solution prepared by mixing the selected parathyroid hormone, a non-volatile buffering agent and an excipient.

The PTH compositions of the present invention incorporate PTH in a therapeutically effective amount, a term used with reference to amounts useful either therapeutically or in medical diagnosis. The particular amount of parathyroid hormone incorporated in the preparation can be pre-determined based on the type of PTH selected and on the intended end-use of the preparation. In one aspect, the compositions are exploited for therapeutic purposes, and particularly for the treatment of osteoporosis and related bone disorders, as well as hypoparathyroidism. In one aspect, such therapy entails administration of the liquid and/or reconstituted lyophilized composition by injection, e.g., a sub-cutaneous injection, in unit doses that reflect the prescribed treatment regimen. In one embodiment, the treatment regimen may include administering recombinant human PTH(1-84) within the range from about 0.01 mg PTH/mL of injected solution to 5 mg PTH/mL of injected solution per patient, with injection volumes being e.g., from about 0.3 mL to about 2.3 mL, or from about 0.5 mL to about 2 mL, or from about 1 mL to about 1.75 mL, or about 1.2 mL, or about 1.3 mL, or about 1.4 mL, or about 1.5 mL, or about 1.6 mL, or about 1.7 mL. Accordingly, in one embodiment, the purified and sterile-filtered PTH is incorporated with the buffering agent and excipients to form an aqueous solution containing PTH in a concentration range from 0.01 mg/mL to 5 mg/mL, or about 0.02 mg/mL to about 2.5 mg/mL, or about 0.025 mg/mL to about 1 mg/mL, or about 0.025 mg/mL to about 0.5 mg/mL, or about 0.025 mg/mL to about 0.25 mg/mL. In one embodiment, PTH is incorporated with the buffering agent and excipients to form an aqueous solution containing PTH in a concentration range, or about 0.025 mg/mL, or about 0.05 mg/mL, or about 0.075 mg/mL, or about 0.1 mg/mL.

Molar equivalents of the substantially equipotent forms of PTH, such as the PTH(1-84) variants and fragments, can be similarly incorporated in place of the human PTH(1-84), if desired.

In some embodiments, the compositions of the invention further comprise a pharmaceutically acceptable excipient and/or carrier. Examples of suitable excipients are provided in Pramanick, S. et al, Excipient Selection in Parenteral Formulation Development, Pharma Times, 2013, 45, 3, 65-77, the contents of which are hereby incorporated by reference in their entirety. Non-limited examples of suitable excipients are presented below.

Surfactant

In some embodiments, formulations disclosed herein further comprise a surfactant. In some embodiments, the surfactant may be selected from poloxamer (e.g., Poloxamer-188), polyethylene glycol, cetyl hydroxyethyl cellulose, hydrophobically modified hydroxyethyl cellulose, polyoxyethylene glycol alkyl ether, polyoxypropylene glycol alkyl ether, glucoside alkyl ether, polyoxyethylene glycol alkylphenol ether, glycerol alkyl ester, polysorbate (e.g., Polysorbate 20 and Polysorbate 80), cocamide monoethaolamine (MEA), cocamide diethanolamine (DEA), dodecyldimethylamine oxide, or any combination thereof. In one embodiment, the surfactant is selected from Poloxamer-188, Polysorbate 20, Polysorbate 80, and polyethylene glycol, and combinations thereof.

In one embodiment, the surfactant is a poloxamer. In one embodiment, the surfactant is Poloxamer-188.

The surfactant may be present at a concentration of about 0.01% to about 20% by weight, about 0.01% to about 15%, about 0.01% to about 10%, about 0.01% to about 5%, about 0.02% to about 4%, about 0.03% to about 3%, about 0.03% to about 1%, about 0.05% to about 0.5%, about 0.1% to about 0.5%, about 0.1% to about 20%, about 0.1% to about 10%, about 0.1% to about 5%, about 0.1% to about 2.5%, 0.1% to about 1%, or about 0.1% to about 0.7%, or about 0.1%, or about 0.2%, or about 0.3%, or about 0.4%, or about 0.5%. In one embodiment, the surfactant is Poloxamer-188, and it is present at about 0.3% w/v of the composition.

Tonicity Agent

In some embodiments, compositions of the present disclosure further comprise a tonicity agent. Tonicity is a measure of the effective osmotic pressure gradient (as defined by the water potential of two solutions) of two solutions separated by a semipermeable membrane. Tonicity is commonly used when describing the response of cells immersed in an external solution. In other words, tonicity is the relative concentration of solutions that determine the direction and extent of diffusion. Body fluids normally have an osmotic pressure that corresponds to that of a 0.9% solution of sodium chloride. A composition (e.g., solution or gel) is considered isotonic when its tonicity is about equal to that of a 0.9% sodium chloride solution (i.e., 290 mOsm/kg). A composition is isotonic with a body fluid solution when the magnitude of the salts is equal between the composition and the physiologic solution. Tonicity equilibrium is reached in physiologic solutions by water moving across the membranes, but the salts staying in their solution of origin. A solution is isotonic with a living cell if there is no net gain or loss of water by the cell, or other changes in the cell, when it is in contact with that solution.

In certain embodiments, a tonicity agent used in the compositions disclosed herein is an electrolyte, mono- or disaccharide, inorganic salt (e.g., sodium chloride, calcium chloride, sodium sulfate, magnesium chloride), a polyol, or a combination thereof. In some embodiments, a tonicity agent is glucose, sucrose, sodium chloride, potassium chloride, calcium chloride, sodium sulfate, magnesium chloride, dextrose, mannitol, glycerol, or any combination thereof. In one embodiment, the tonicity agent is selected from sodium chloride, sucrose, and glycerol, or combinations thereof. In one embodiment, the tonicity agent is sucrose. In one embodiment, the tonicity agent is sodium chloride. In one embodiment, the tonicity agent is glycerol.

The tonicity agent may be present at any concentration necessary to achieve isotonic conditions. In some embodiments, the tonicity agent may be present at a concentration of about 0.01% to about 50%, about 0.01% to about 40%, about 0.01% to about 30%, about 0.01% to about 20%, about 0.02% to about 20%, about 0.03% to about 20%, about 0.05% to about 15%, about 0.1% to about 10%, about 0.1% to about 20%, about 0.1% to about 15%, about 0.1% to about 9%, about 0.2% to about 10%, 0.5% to about 10%, or about 1% to about 10%, or about 1%, or about 2%, or about 3%, or about 4%, or about 5%, or about 6%, or about 7%, or about 8%, or about 9% w/v of the composition. In one embodiment, the tonicity agent is sucrose, and it is present at about 0.2% to about 20% of the composition, or at about 8.5% w/v of the composition. In one embodiment, the tonicity agent is glycerol, and it is present at about 0.2% to about 20% of the composition, or at about 2.3% w/v of the composition. In one embodiment, the tonicity agent is sodium chloride, and it is present at about 0.2% to about 20% of the composition, or at about 0.8% w/v of the composition.

Preservative

In some embodiments, compositions of the present disclosure are sterile and preservative-free. In other embodiments, compositions of the present disclosure optionally comprise a preservative. In particular embodiments, a preservative is a paraben-free preservative. Parabens are a series of parahydroxybenzoates or esters of parahydroxybenzoic acid and are known to cause cytokine release and irritation and have been linked to several types of cancer. Examples of parabens include methyl paraben, ethyl paraben, propyl paraben, butyl paraben, heptyl paraben, isobutyl paraben, isopropyl paraben, benzyl paraben, and their sodium salts.

Exemplary paraben-free preservatives include methylphenol (cresol), including 3-methylphenol (meta-cresol or m-cresol), phenol, phenethyl alcohol, caprylyl glycol, phenoxyethanol, a sorbate, potassium sorbate, sodium sorbate, sorbic acid, sodium benzoate, benzoic acid, acemannan, oleuropein, carvacrol, cranberry extract, gluconolactone, green tea extract, Helianthus annuus seed oil, Lactobacillus ferment, Usnea barbata extract, polyaminopropyl biguanide, polyglyceryl-3 palmitate, polyglyceryl-6 caprylate, pomegranate extract, Populus tremuloides bark extract, resveratrol, Rosmarinus officinalis leaf extract, benzyl alcohol, or any combination thereof.

In one embodiment, a preservative is selected from m-cresol, phenol, benzyl alcohol, sodium benzoate, and propyl paraben, and combinations thereof. In one embodiment, a preservative comprises m-cresol.

In some embodiments, compositions of this disclosure may comprise a preservative at a concentration of about 0.005% to about 10% by weight, about 0.005% to about 5%, about 0.01% to about 5%, about 0.02% to about 4%, about 0.03% to about 3%, about 0.05% to about 2%, about 0.1% to about 1%, about 0.2% to about 0.5%, about 0.01% to about 10%, about 0.01% to about 5%, about 0.01% to about 2.5%, about 0.01% to about 1%, about 0.01% to about 0.5%, about 0.1% to about to about 10%, about 0.1% to about 5%, about 0.1% to about 2.5%, about 0.1% to about 1%, about 0.1% to about 0.5%, or about 0.1%, or about 0.2%, or about 0.3%, or about 0.4%, or about 0.5% w/v of the composition. In one embodiment, the preservative is m-cresol present at about 0.03% to about 3% of the composition. In one embodiment, m-cresol is present at 0.3% of the composition.

Pharmaceutically Acceptable Buffer

In some embodiments, compositions of the present invention may comprise a pharmaceutically acceptable buffer by incorporating a buffering agent. In one embodiment, buffering agents incorporated in the present compositions are selected from those capable of buffering the preparation to a pH within a physiologically acceptable range. A pH that is physiologically acceptable is that which causes either no, or minimal, patient discomfort when the formulation is administered, and can thus vary depending on the mode of administration. For preparations that will be diluted prior to administration, such as by dissolution in a stock infusion solution, the pH of the preparation per se can vary widely, e.g., from about pH 3 to about pH 9. Where the preparation is to be administered directly after reconstitution, the PTH preparation is buffered to within the pH range from 3.5 to 7.5. Suitable buffers are accordingly those pharmaceutically acceptable agents that can buffer the pH of the preparation to within the target pH range, and include acetate buffers, phosphate buffers, L-Histidine buffers, succinate buffers.

Although any pharmaceutically acceptable buffers may be suitable for formulations according to the invention, it has been surprisingly found that the nature of the buffering agent has a large effect on the stability of rhPTH solutions.

For example, citrate buffer, which is currently used in NATPARA®, results in rhPTH protein particulate formation at as little as 24 hours of agitation at ambient conditions. However, solutions of rhPTH prepared with acetate, phosphate, and L-Histidine buffers remain clear, colorless and free of visible particles for 24 hours of agitation.

To provide in-use stable formulations of parathyroid hormone in accordance with the invention, the selected buffering agent is incorporated to yield a final pH within the range from 3.5 to 6.5, and the buffer is present at a concentration of about 5 mM to about 50 mM. In some embodiments of the invention, the pH rendered by the buffering agent is in the range from 3.8 to 6.2, and the buffer concentration about 10 mM to about 30 mM. In one embodiment, the pH of the formulation is 5.5. In one embodiment, the pH of the formulation is 4.3. In one embodiment the buffer is acetate buffer present at a concentration of about 20 mM. In one embodiment the buffer is L-Histidine buffer present at a concentration of about 20 mM. In one embodiment the buffer is succinate buffer present at a concentration of about 20 mM.

Antioxidant

In some embodiments, the formulations of the invention may further comprise one or more antioxidants to provide oxidative stability to the rhPTH protein during the in-use period. Antioxidants that may be suitable may include, without limitation, acetone sodium bisulfite, argon, ascorbyl palmitate, ascorbate (salt/acid), bisulfite sodium, butylated hydroxy anisole (BHA), butylated hydroxy toluene (BHT), cysteine/cysteinate HCl, dithionite sodium (NA hydrosulfite, Na sulfoxylate), gentisic acid, gentisic acid ethanolamine, glutamate monosodium, glutathione, formaldehyde sulfoxylate sodium, metabisulfite potassium, methionine, monothioglycerol (thioglycerol), nitrogen, propyl gallate, sulfite sodium, tocopherol alpha, alpha tocopherol hydrogen succinate, thioglycolate sodium, or combinations of two or more thereof. In one embodiment, the antioxidant may be methionine.

The antioxidant may be present at any concentration necessary to achieve oxidative stability of the formulation. In some embodiments, the antioxidant may be present at a concentration of about 0.0001% to about 20% by weight, about 0.001% to about 10%, about 0.01% to about 5%, about 0.01% to about 2%, about 0.02% to about 2%, about 0.03% to about 2%, about 0.05% to about 1.5%, about 0.1% to about 1% w/v. In one embodiment, the antioxidant is methionine present in an amount from about 0.015% to about 1.5% of the composition. In one embodiment, the antioxidant is methionine present in an amount of about 0.15% w/v of the composition.

Novel Lyophilized Formulations

In one aspect, the parathyroid hormone compositions of the present invention are provided in a lyophilized powder form containing not more than 3% water by weight, that results from the freeze-drying of a sterile, aqueous hormone solution prepared by mixing the selected parathyroid hormone, a non-volatile buffering agent and an excipient.

In one embodiment of the invention, the lyophilized compositions are provided in a form that yields a unit dose of about 0.05 mg/mL to about 0.15 mg/mL recombinant human PTH(1-84) upon reconstitution into about 1 to 1.5 mL (0.7-1.8 mL) of the reconstitution vehicle, and the vials are accordingly loaded with about 1 to 1.5 mL of the aqueous PTH preparation, for subsequent freeze-drying.

In one embodiment of the invention, the PTH preparation subjected to freeze-drying comprises from 25 to 250 μg/mL of human PTH(1-84), about 0.3% to about 30% w/v bulking agent, about 0.2% to about 20% w/v tonicity agent, and a physiologically acceptable buffering agent in an amount capable of buffering the preparation to within the range from 3.5 to 6.5 upon reconstitution in sterile water. In specific embodiments of the invention, the buffering agent is incorporated in an amount sufficient to buffer the pH to 5.5±0.3, or 4.3±0.3.

Bulking Agent

In some embodiments, novel lyophilized formulations may further comprise one or more bulking agent for optimal cake structure and appearance. Bulking agents that may be suitable include compatible carbohydrates, polypeptides, amino acids or combinations thereof. Suitable carbohydrates may include monosaccharides such as galactose, D-mannose, sorbose, and the like; disaccharides, such as lactose, trehalose, and the like; cyclodextrins, such as 2-hydroxypropyl-β-cyclodextrin; polysaccharides, such as raffinose, maltodextrins, dextrans, and the like; and alditols, such as mannitol, xylitol, and the like. Suitable polypeptides include aspartame. Amino acids include alanine and glycine. In one embodiment, novel lyophilized formulations may comprise one or more bulking agent selected from mannitol, glycine, poly(ethylene glycols), ammonium sulfate, sucrose, trehalose, and combinations thereof. In one embodiment, novel lyophilized formulations may comprise mannitol.

The bulking agent may be present at any concentration necessary to achieve the optimal structure and appearance of the lyophilized powder. In some embodiments, the bulking agent may be present at a concentration of about 0.01% to about 50% by weight, about 0.01% to about 40%, about 0.01% to about 30%, about 0.01% to about 20%, about 0.02% to about 20%, about 0.03% to about 20%, about 0.05% to about 15%, about 0.1% to about 10%, about 0.1% to about 20%, about 0.1% to about 15%, about 0.1% to about 9%, about 0.2% to about 10%, 0.5% to about 10%, or about 1% to about 10%, or about 1%, or about 2%, or about 3%, or about 4%, or about 5%, or about 6%, or about 7%, or about 8%, or about 9% w/v of the composition. In one embodiment, the bulking agent is mannitol, and it is present at about 0.2% to about 20% of the composition, or at about 2% to about 8% of the composition, or at about 3% w/v of the composition, or at about 4% of the composition.

Cryoprotectant

In some embodiments, novel lyophilized formulations may further comprise one or more cryoprotectants to provide stability to the rhPTH protein during the freeze-drying process and product storage. Cryoprotectants that may be suitable include compatible carbohydrates, such as sugars and polyols. Suitable carbohydrates may include glucose, sucrose, trehalose, ethylene glycol, propylene glycol, 2-methyl-2,4-pentaglycol, and glycerol. In one embodiment, novel lyophilized formulations may comprise one or more cryoprotectants selected from sucrose, glycine, mannitol, disaccharides, poly(ethylene glycols) and combinations thereof. In one embodiment, novel lyophilized formulations may comprise sucrose.

The cryoprotectant may be present at any concentration necessary to achieve stability of the lyophilized powder. In some embodiments, the cryoprotectant may be present at a concentration of about 0.01% to about 50% by weight, about 0.01% to about 40%, about 0.01% to about 30%, about 0.01% to about 20%, about 0.02% to about 20%, about 0.03% to about 20%, about 0.05% to about 15%, about 0.1% to about 10%, about 0.1% to about 20%, about 0.1% to about 15%, about 0.1% to about 9%, about 0.2% to about 10%, 0.5% to about 10%, or about 1% to about 10%, or about 1%, or about 2%, or about 3%, or about 4%, or about 5%, or about 6%, or about 7%, or about 8%, or about 9% w/v of the composition. In one embodiment, the cryoprotectant is sucrose, and it is present at about 0.2% to about 20% of the composition, or at about 1% to about 8% of the composition, or at about 2% w/v of the composition, or at about 3% of the composition.

Dosage Forms

The compositions may be provided in single or multiple dose injectable form, for example in the form of a pen. The compositions may, as already mentioned, be prepared by any suitable pharmaceutical method which includes a step in which the active ingredient and the carrier (which may consist of one or more additional ingredients) are brought into contact.

In certain embodiments the pharmaceutical composition may be provided together with a device for application, for example together with a syringe, an injection pen or an auto-injector, e.g., a Q-cliq pen. Such devices may be provided separate from a pharmaceutical composition or prefilled with the pharmaceutical composition.

### EXAMPLES

The following examples illustrate specific aspects of the instant description. The examples should not be construed as limiting, as the examples merely provide specific understanding and practice of the embodiments and their various aspects.

The formulations were prepared in the following manner: rhPTH(1-84) drug substance (active pharmaceutical ingredient) was exchanged against the respective base formulation buffer using a dialysis method commonly known by those of skill in the art. Solution pH adjustment was further made, if needed, with acid or base stock solutions. Stock solutions of excipients were prepared separately in the base buffer, and were mixed with the dialyzed peptide solution to achieve the final formulations with the desired peptide and excipient concentrations. The formulations were sterile filtered and filled either in glass vials or in glass cartridges. Liquid formulations were stoppered and crimped, followed by storage. Formulations meant for lyophilization were exposed to a pre-programmed lyophilization cycle consisting of freezing, annealing, primary drying and secondary drying steps, followed by stoppering and crimping.

### Example 1: Compositions of Novel Liquid Formulations of rhPTH

Table 1, below, summarizes exemplary embodiments of novel liquid formulations of rhPTH according to the invention. As shown it Table 1, Liquid Formulations #1 to #3 have the following composition:

Liquid Formulation #1

0.35 to 1.40 mg/mL rhPTH;

20 mM Acetate Buffer;

10 mM Methionine;

130 mM Sodium Chloride;

0.3% w/v Poloxamer-188, and

0.3% w/v m-cresol in water.

Liquid Formulation #2

0.35 to 1.40 mg/mL rhPTH;

20 mM Acetate Buffer;

10 mM Methionine;

8.5% w/v Sucrose;

0.3% w/v Poloxamer-188, and

0.3% w/v m-cresol in water.

Liquid Formulation #3

0.35 to 1.40 mg/mL rhPTH;

20 mM Acetate Buffer;

10 mM Methionine;

2.3% v/v Glycerol;

0.3% w/v Poloxamer-188, and

0.3% w/v m-cresol in water.

The pH of Liquid Formulations #1 to #3 is 5.5.

TABLE 1
Compositions of Novel Liquid Formulations or rhPTH

| Liquid | |
|---|---|
| Formulation # | Composition |
| 1 | rhPTH (0.35 to 1.40 mg/mL) in pH 5.5, 20 mM |
| | Acetate Buffer, 10 mM Methionine, 130 mM |
| | Sodium Chloride, 0.3% w/v Poloxamer-188, |
| | and 0.3% w/v m-cresol in water |
| 2 | rhPTH (0.35 to 1.40 mg/mL) in pH 5.5, 20 mM |
| | Acetate Buffer, 10 mM Methionine, 8.5% w/v |
| | Sucrose, 0.3% w/v Poloxamer-188, |
| | and 0.3% w/v m-cresol in water |
| 3 | rhPTH (0.35 to 1.40 mg/mL) in pH 5.5, 20 mM |
| | Acetate Buffer, 10 mM Methionine, 2.3% v/v |
| | Glycerol, 0.3% w/v Poloxamer-188, |
| | and 0.3% w/v m-cresol in water |

### Example 2: Compositions of Novel Lyophilized Powder Formulations of rhPTH

Table 2, below, summarizes exemplary embodiments of novel lyophilized powder formulations of rhPTH according to the invention. As shown it Table 2, Lyophilized Formulations #1 to #3 have the following composition:

Lyophilized Formulation #1

0.35 to 1.40 mg/mL rhPTH;

20 mM L-Histidine Buffer;

4% w/v Mannitol, and

2% Sucrose in water.

Lyophilized Formulation #2

0.35 to 1.40 mg/mL rhPTH;

20 mM L-Histidine Buffer;

10 mM Methionine;

4% w/v Mannitol;

2% w/v Sucrose, and

0.3% w/v Poloxamer-188 in water.

Lyophilized Formulation #3

0.35 to 1.40 mg/mL rhPTH;

20 mM Succinate Buffer;

10 mM Methionine;

3% w/v Mannitol, and

3% w/v Sucrose in water.

The pH of Lyophilized Formulations #1 and #2 is 5.5. The pH of Lyophilized Formulation #3 is 4.3

TABLE 2
Compositions of Novel Lyophilized Formulations or rhPTH

| Lyophilized | |
|---|---|
| Formulation # | Composition |
| 1 | rhPTH (0.35 to 1.40 mg/mL) in pH 5.5, 20 mM |
| | L-Histidine Buffer, 4% w/v Mannitol, and |
| | 2% w/v Sucrose in water |
| 2 | rhPTH (0.35 to 1.40 mg/mL) in pH 5.5, 20 mM |
| | L-Histidine Buffer, 10 mM Methionine, 4% w/v |
| | Mannitol, 2% w/v Sucrose, and 0.3% w/v |
| | Poloxamer-188 in water |
| 3 | rhPTH (0.35 to 1.40 mg/mL) in pH 4.3, 20 mM |
| | Succinate Buffer, 3% w/v Mannitol, and |
| | 3% w/v Sucrose in water |

### Example 3: Agitation Studies for rhPTH Formulated in Different Buffers

Agitation (shaking) in actual drug product storage container/closures or in small scale representative primary containers is often applied in the development of protein pharmaceuticals, serving as a test of stability under physical stress conditions also occurring in the real process. The overall purpose of these “stress tests” is to accelerate protein degradation/aggregation that could otherwise take place at a much slower rate, thereby enhancing experimental throughput to speed up determination of critical process parameters of stability. Results are useful to determine critical parameters for formulation development.

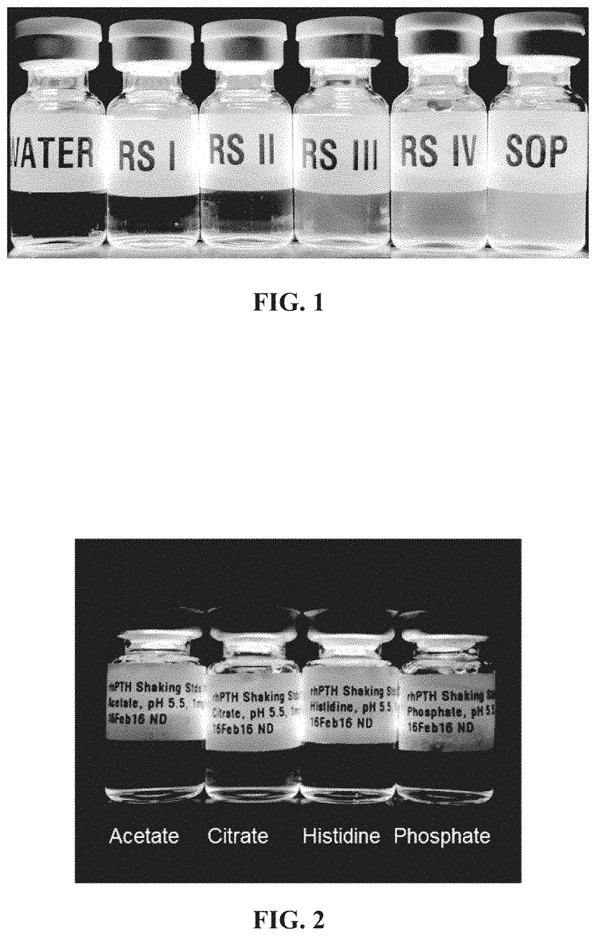

Table 3, below, and FIG. 2 show appearance data from agitation studies where rhPTH was formulated with different buffers. rhPTH was formulated in different buffers (10 mM) with sodium chloride (140 mM) in 2R glass vials. The agitation studies were performed using an orbital shaker at 220 rpm, with vials in horizontal position, at ambient conditions for at least 48 hours. On regular agitation intervals, the visual appearance of agitated samples were compared to reference suspensions (RSI-IV) and Standard of Opalescence (SOP) according to standard procedure outlined, e.g., in European Pharmacopoeia 5.0, 2.2.1. Clarity and Degree of Opalescence in Liquids. FIG. 1 shows opalescence of reference suspensions RSI-IV and SOP. Water is provided for comparison. FIG. 2 shows appearance of the suspensions of rhPTH formulated in different buffers.

TABLE 3
Appearance data from agitation studies for rhPTH
(formulated in different buffers)
at T 0 and after 4 h, 8 h, 24 h, and 48 h of agitation

| Formulation | T 0 | 4 h | 8 h | 24 h | 48 h |
|---|---|---|---|---|---|
| Acetate | Clear, | Clear, | Clear, | Clear, | RS II |
| | colorless, | colorless, | colorless, | colorless, | |
| | and free | and free | and free | and free | |
| | of visible | of visible | of visible | of visible | |
| | particles | particles | particles | particles | |
| Citrate | Clear, | Clear, | Clear, | RS I | >SOP |
| | colorless, | colorless, | colorless, | | |
| | and free | and free | and free | | |
| | of visible | of visible | of visible | | |
| | particles | particles | particles | | |
| Histidine | Clear, | Clear, | Clear, | Clear, | RS III |
| | colorless, | colorless, | colorless, | colorless, | |
| | and free | and free | and free | and free | |
| | of visible | of visible | of visible | of visible | |
| | particles | particles | particles | particles | |
| Phosphate | Clear, | Clear, | Clear, | Clear, | RS II |
| | colorless, | colorless, | colorless, | colorless, | |
| | and free | and free | and free | and free | |
| | of visible | of visible | of visible | of visible | |
| | particles | particles | particles | particles | |

*rhPTH (1 mg/mL) was formulated with 10 mM of buffer species and 140 mM sodium chloride;
RS: Reference suspension;
SOP: Standard of opalescence

As shown in Table 3 and FIG. 2, acetate buffer shows the best stability against agitation induced particulate formation in rhPTH, followed by phosphate buffer, followed by L-Histidine buffer. All three buffers show better stability than citrate buffer, which is currently used in the NATPARA® formulation.

### Example 4: Agitation Studies for rhPTH in Novel Liquid Formulations

Table 4, below, shows appearance data from agitation studies for various liquid formulations of rhPTH according to the invention. Liquid Formulations #1-#3 were formulated in dual-chamber cartridges, and agitation studies were performed at ambient conditions (220 rpm, orbital shaking). On regular agitation intervals, visual appearance of agitated samples was compared to reference suspensions (RSI-IV) and Standard of Opalescence (SOP) according to standard procedure. Data for the commercial NATPARA® formulation is also provided for comparison. The opalescence at different time points is summarized in Table 4.

TABLE 4
Appearance data from agitation studies for rhPTH (formulated in
different buffers) at T 0 and after 5 h, 24 h, 48 h, and 72 h of agitation

| Formulation | T 0 | 5 h | 24 h | 48 h | 72 h |
|---|---|---|---|---|---|
| Liquid Formulation # 1: | Clear and free of visible particles (W and RSI) | | | | |
| rhPTH (1 mg/mL) in pH 5.5, 20 mM | | | | | |
| Acetate Buffer, 25 mM Methionine^{#}, | | | | | |
| 130 mM Sodium Chloride, | | | | | |
| 0.3% w/v Poloxamer-188, | | | | | |
| and 0.3% w/v m-cresol in water | | | | | |
| Liquid Formulation # 2: | Clear and free of visible particles (W and RSI) | | | | |
| rhPTH (1 mg/mL) in pH 5.5, 20 mM | | | | | |
| Acetate Buffer, 25 mM Methionine^{#}, 8.5% | | | | | |
| w/v Sucrose, 0.3% w/v Poloxamer-188, | | | | | |
| and 0.3% w/v m-cresol in water | | | | | |
| Liquid Formulation # 3: | Clear and free of visible particles (W and RSI) | | | | |
| rhPTH (1 mg/mL) in pH 5.5, 20 mM | | | | | |
| Acetate Buffer, 25 mM Methionine^{#}, 2.3% | | | | | |
| v/v Glycerol, 0.3% w/v Poloxamer-188, | | | | | |
| and 0.3% w/v m-cresol in water | | | | | |
| Natpara ® | Clear and free | RSIV | >SOP | | |
| | of visible particles | and | | | |
| | (RSII and RSIII) | SOP | | | |

W: Water;
RS: Reference suspension;
SOP: Standard of opalescence;
^{#}Methionine concentration used in these studies was 25 mM instead of 10 mM used in the novel liquid formulations (methionine concentration was observed not to have any impact on the rhPTH particulate formation when agitated in 2R vials).

As shown in Table 4, all three novel liquid formulations #1-#3 stayed clear and free of visible particles for at least 72 hours. In contrast, the current commercial formulation showed presence of significant particles and higher opalescence in as early as 5 hours of agitation period.

### Example 5: Agitation Studies for rhPTH in Novel Lyophilized Formulations

Table 5, below, shows appearance data from agitation studies for various reconstituted lyophilized formulations of rhPTH according to the invention. Lyophilized Formulations #1-#3 were formulated in dual-chamber cartridges, and agitation studies were performed at ambient conditions (220 rpm, orbital shaking). On regular agitation intervals, visual appearance of agitated samples was then compared to reference suspensions (RSI-IV) and Standard of Opalescence (SOP) according to standard procedure. Data for the commercial NATPARA® formulation is also provided for comparison. The opalescence at different time points is summarized in Table 5.

TABLE 5
Appearance data from agitation studies for rhPTH (formulated in
different buffers) at T0 and after 5 h, 24 h, 32 h, 48 h, and 90 h of agitation

| Formulation* | T0 | 5 h | 24 h | 32 h | 48 h | 90 h |
|---|---|---|---|---|---|---|
| Lyo Formulaton # 1: rhPTH (1 mg/mL) in pH 5.5, 20 mM L-Histidine | Clear, colorless, and free of visible particles | | | | >SOP | |
| Buffer, 4% w/v Mannitol, and 2% w/v Sucrose in water | | | | | | |
| Lyo Formulation # 2^{#}: rhPTH (1 mg/mL) in pH 5.5, 20 mM L-Histidine | Clear, colorless, and free of visible particles | | | | | |
| Buffer, 4% w/v Mannitol, 2% w/v Sucrose, and 0.3% w/v Poloxamer-188 | | | | | | |
| in water | | | | | | |
| Lyo Formulation # 3: rhPTH (1 mg/mL) in pH 4.3, 20 mM Succinate | Clear, colorless, and free of visible particles | | | | | |
| Buffer, 3% w/v Mannitol, and 3% w/v Sucrose in water | | | | | | |
| Natpara ® | Clear and free of | RSIV and | >SOP | | | |
| | visible particles | SOP | | | | |
| | (RSII and RSIII) | | | | | |

*Formulation was reconstituted with 0.3% w/v m-cresol prior to agitation; RS: Reference suspension; SOP: Standard of opalescence; #Methionine (10 mM) was not incorporated in this formulation during shaking studies (methionine concentration was observed not to have any impact on the rhPTH particulate formation when agitated in 2R vials with novel liquid formulations).

As the above Examples show, it can be observed that the novel lyophilized formulations significantly improve the physical stability of rhPTH. As shown above, the novel formulations of rhPTH stay clear, colorless, and free of visible particles for at least 24 hours, and/or at least 48 hours, and/or at least 72 hours, and/or at least 90 hours.

### Example 6: In-Use Stability Studies for Liquid and Lyophilized Formulations

Table 6, below, shows appearance data from in-use studies for a liquid formulation according to an embodiment of the invention, as exemplified by Liquid Formulation #2, and a reconstituted lyophilized formulation of rhPTH according to an embodiment of the invention, as exemplified by Lyophilized Formulation #2.

TABLE 6
In-use appearance data for Liquid Formulation #2 and Lyophilized Formulation
#2 at in-use period of 1 day, 7 days, 14 days, and 21 days

| Formulation | Day-1 | Day-7 | Day-14 | Day-21 |
|---|---|---|---|---|
| Liquid Formulation #2: | Clear and free of | Clear and free of | Clear and free of | Clear and free of |
| rhPTH (1 mg/mL) in pH | visible particles | visible particles | visible particles | visible particles |
| 5.5, 20 mM Acetate | with opalescence | with opalescence | with opalescence | with opalescence |
| Buffer, | between RSI and | between RSI and | between RSI and | between RSI and |
| 10 mM Methionine, 8.5% | RSII | RSII | RSII | RSII |
| w/v Sucrose, 0.3% w/v | | | | |
| Poloxamer-188, and 0.3% | | | | |
| w/v m-cresol in water | | | | |
| Lyophilized | Clear and free of | Clear and free of | Clear and free of | Clear and free of |
| Formulation # 2: | visible particles | visible particles | visible particles | visible particles |
| rhPTH (1 mg/mL) in pH | with opalescence | with opalescence | with opalescence | with opalescence |
| 5.5, | between RSI and | between RSI and | between RS I and | between RSI and |
| 20 mM L-Histidine | RSII | RSII | RSII | RSII |
| Buffer, | | | | |
| 10 mM Methionine, | | | | |
| 4% w/v Mannitol, | | | | |
| 2% w/v Sucrose, | | | | |
| 0.3% w/v Poloxamer-188, | | | | |
| and 0.3% w/v m-cresol* | | | | |
| in water | | | | |

*Samples were reconstituted with 0.3% w/v m-cresol containing water for injection prior to start of the in-use

As Table 6, above, shows, the novel liquid and lyophilized formulations of rhPTH, as exemplified by Liquid Formulation #2 and Lyophilized Formulation #2, stay clear, colorless, and free of visible particles for the in-use period of at least 1 day, or at least 7 days, or at least 14 days, or at least 21 days.

### Example 7: Solution pH Screening for rhPTH for Optimal Physicochemical Stability

Recombinant human parathyroid hormone was formulated in 10 mM citric acid buffer with 140 mM sodium chloride in a solution pH range of 3.5 to 7.5 with a 0.5 unit pH interval. The samples were aliquoted in 2 mL Type I borosilicate glass vials and placed on stability at temperature conditions of 5±3° C. (5° C.), 25±2° C. (25° C.), and 40±2° C. (40° C.). At pre-defined intervals, samples were pulled, observed for appearance, and analyzed for rhPTH stability using the chromatographic assays in place (Size Exclusion Chromatography (SEC) and Reversed-Phase Chromatography (RP-HPLC)) with some modifications.

The supplied drug substance material was thawed and dialyzed against respective pH buffer solutions in 2 kDa molecular weight cut off (MWCO) dialysis cassette. The dialysis was performed at 5±3° C. and included at least 3 cycles of buffer exchanges over a period of ˜24 hours. Post dialysis, samples were assayed for pH and adjusted if necessary with 0.2 N sodium hydroxide. A280 measurements were performed and rhPTH concentration was calculated using an extinction coefficient of 0.584 (mL·mg)⁻¹cm⁻¹. Final solution preparation was done aseptically in a laminar flow hood. For each solution pH, rhPTH was prepared at a concentration of 1.0 mg/mL by using the respective buffer as the dilution medium. The prepared sample was filtered via a 0.22 μm PVDF filter, filled at 1.5 mL volume in 2 mL Type I borosilicate glass vials, followed by stoppering/crimping.

Each vial was observed for solution appearance in a light box. Baseline samples were separated, aliquoted in polypropylene tubes, and stored at −80° C. Remaining vials were incubated at 5, 25, and 40° C. At a predefined interval, sample vials were pulled from each incubation condition, observed for appearance, aliquoted in polypropylene tubes, and stored at −80° C. until analysis. Samples were tested for physical and chemical changes using assays validated for Natpara®, including SEC and RP-HPLC with some modifications in injection volume and injection sequence.

To demonstrate physical stability, tables 7 and 8 show the appearance results for the rhPTH stability samples stored at 40 and 25° C., respectively, for 6 months. Opalescence against a reference suspension is noted where measured. White flocculant like particles were visible in pH 7.0 and 7.5 samples within 2 weeks of storage at 40° C. This particle formation appeared to progress from the basic to acidic side of the solution pH with time. By 3 months, most of the samples stored at 40° C. had particles. Samples stored at 25° C. showed the same trend of particle formation as observed at 40° C. but with slower kinetics. It was also noticed that the size of particles was different depending on the solution pH. Samples formulated in the pH range of 6.5-7.5 had flocculants, while those at lower pH had fine particles. Samples stored at 5° C. had an initial appearance of clear, colorless, free of visible of particles which did not change over the course of 6 months.

TABLE 7
Appearance results for rhPTH pH screen samples
stored at 40° C. temperature conditions

| Solution | | | | | |
|---|---|---|---|---|---|
| pH | T 0 | 2 w | 1 m | 3 m | 6 m |
| 3.5 | CCFVP | CCFVP | CCFVP | CCFVP | Fine particles |
| | | | | | (RSII and RSIII) |
| 4.0 | CCFVP | CCFVP | CCFVP | Fine particles | Fine particles |
| | | | | (solution had slight | (RSIV) |
| | | | | opalescence) | |
| 4.5 | CCFVP | CCFVP | CCFVP | Fine particles | Fine particles |
| | | | | (solution had slight | (RSIV) |
| | | | | opalescence) | |
| 5.0 | CCFVP | CCFVP | CCFVP | Fine particles | Fine particles |
| | | | | (solution had slight | (RSIV) |
| | | | | opalescence) | |
| 5.5 | CCFVP | CCFVP | CCFVP | Fine particles | Fine particles |
| | | | | (solution had slight | (RSIV) |
| | | | | opalescence) | |
| 6.0 | CCFVP | CCFVP | CCFVP | White flocculants | Large floccunants |
| | | | | (solution had slight | (RSIII) |
| | | | | opalescence) | |
| 6.5 | CCFVP | CCFVP | White flocculants | White flocculants | Large floccunants |
| | | | (solution had slight | (solution had slight | (RSIII) |
| | | | opalescence) | opalescence) | |
| 7.0 | CCFVP | White flocculants | White flocculants | White flocculants | Large floccunants |
| | | (solution had slight | (solution had slight | (solution had slight | (RSIII) |
| | | opalescence) | opalescence) | opalescence) | |
| 7.5 | CCFVP | White flocculants | White flocculants | White flocculants | Large floccunants |
| | | (with clear solution | (with clear solution | (solution had slight | (RSIII) |
| | | backgrounds) | backgrounds) | opalescence) | |

CCFVP: Clear, colorless, free of visible particles;
RS: Reference Suspension

TABLE 8
Appearance results for rhPTH pH screen samples stored
at 25° C. temperature conditions

| Solution | | | | |
|---|---|---|---|---|
| pH | T 0 | 1 m | 3 m | 6 m |
| 3.5 | CCFVP | CCFVP | CCFVP | CCFVP |
| 4.0 | CCFVP | CCFVP | CCFVP | CCFVP |
| 4.5 | CCFVP | CCFVP | CCFVP | CCFVP |
| 5.0 | CCFVP | CCFVP | CCFVP | CCFVP |
| 5.5 | CCFVP | CCFVP | CCFVP | CCFVP |
| 6.0 | CCFVP | CCFVP | CCFVP | CCFVP |
| 6.5 | CCFVP | CCFVP | CCFVP | No visible particles |
| | | | | (RSI and RSII) |
| 7.0 | CCFVP | CCFVP | Fine particles | No visible particles |
| | | | to flocculants | (RSIII and RSIV) |
| | | | (solution had slight | |
| | | | opalescence) | |
| 7.5 | CCFVP | CCFVP | White flocculants | White flocculants |
| | | | (with clear solution | (RSIII and RSIV) |
| | | | background) | |

CCFVP: Clear, colorless, free of visible particles;
RS: Reference Suspension

To demonstrate chemical stability, Table 9 provides the protein concentration data for the stability samples stored at 40 and 25° C., respectively. Samples were thoroughly centrifuged (17,000 g for 5 minutes) and supernatants were used for A280 measurements. Appropriate light scattering corrections were made (A320 subtraction). At 40° C., a drop in protein concentration roughly correlated with sample tendency to form particles during storage. No change in protein concentration over time was observed for samples stored at 25° C. (Table 9) and 5° C.

TABLE 9
Protein concentration results (mg/mL) for rhPTH pH screen
samples stored under different temperature conditions

| | | 40° C. | | | 25° C. | |
|---|---|---|---|---|---|---|
| Solution pH | T0 | 1 m | 3 m | 6 m | 3 m | 6 m |
| 3.5 | 1.04 | 1.04 | 0.98 | 0.87 | 0.97 | 0.98 |
| 4.0 | 1.06 | 1.00 | 0.97 | 0.59 | 1.00 | 0.99 |
| 4.5 | 1.03 | 1.01 | 0.94 | 0.75 | 1.01 | 1.00 |
| 5.0 | 1.15 | 1.11 | 0.93 | 0.76 | 1.02 | 1.02 |
| 5.5 | 1.10 | 1.02 | 0.95 | 0.58 | 0.97 | 0.97 |
| 6.0 | 1.13 | 1.10 | 1.05 | 0.97 | 1.00 | 1.02 |
| 6.5 | 1.02 | 1.09 | 0.96 | 0.87 | 0.98 | 0.98 |
| 7.0 | 1.03 | 0.97 | 0.91 | 0.73 | 0.97 | 0.97 |
| 7.5 | 1.02 | 0.95 | 0.65 | 0.53 | 0.98 | 0.98 |

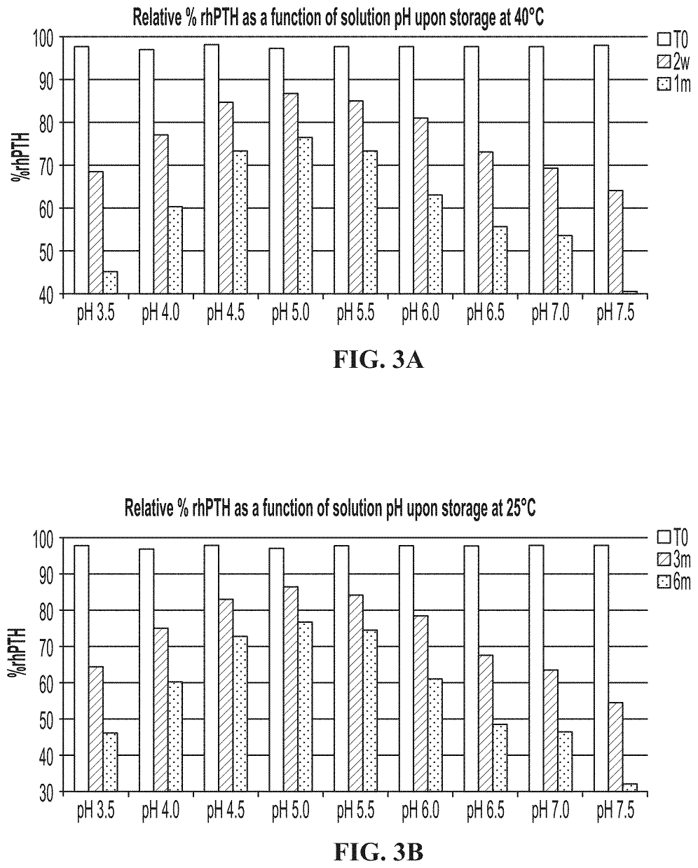

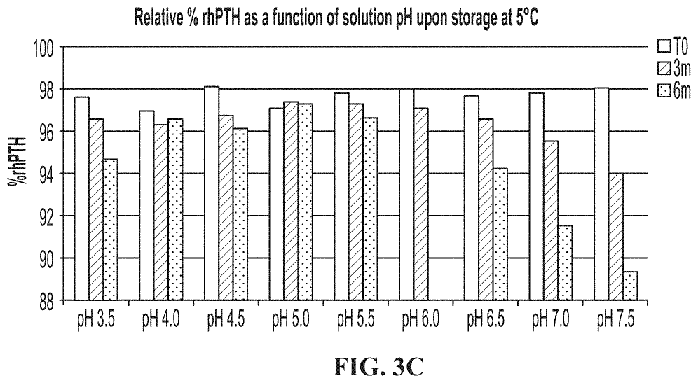

To further demonstrate chemical stability, FIGS. 3-8 shows the RP-HPLC data for the rhPTH and associated impurities for the pH screen samples stored at 40, 25, and 5° C. for up to 6 months. For the 40° C. storage condition, only data for up to 1 month is presented, as the samples were too degraded afterwards to perform peak integration.

Main peak: A bell shape trend for the main peak was observed for all storage temperature, with maximum peak recovery around pH ˜5.0-6.0, as shown in FIGS. 3A-3C.

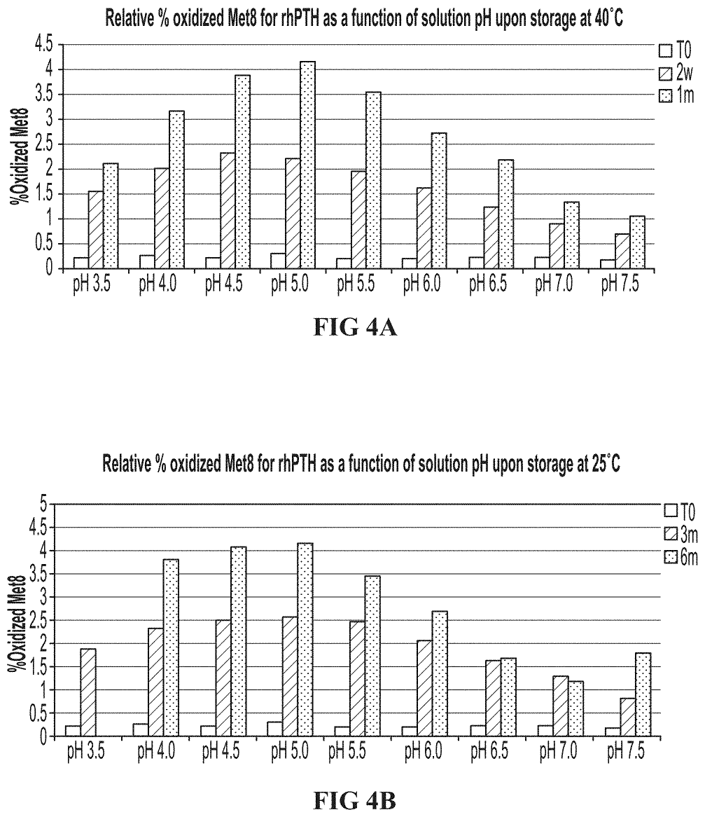

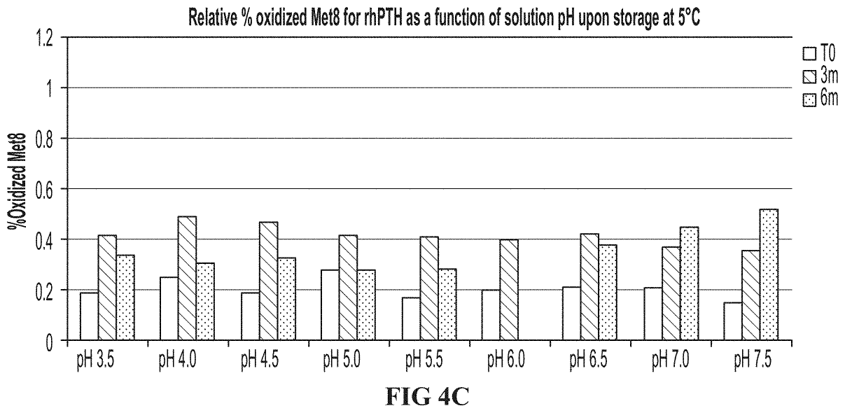

Oxidized Met8: Oxidation of Met8 was also observed to follow a bell shape trend (as with the main peak), where the maximum Met8 oxidation was seen in the pH range of −4.0-5.5 upon storage at 40 and 25° C. At 5° C., no trend was observed up to 6 months of storage, as shown in FIGS. 4A-4C.

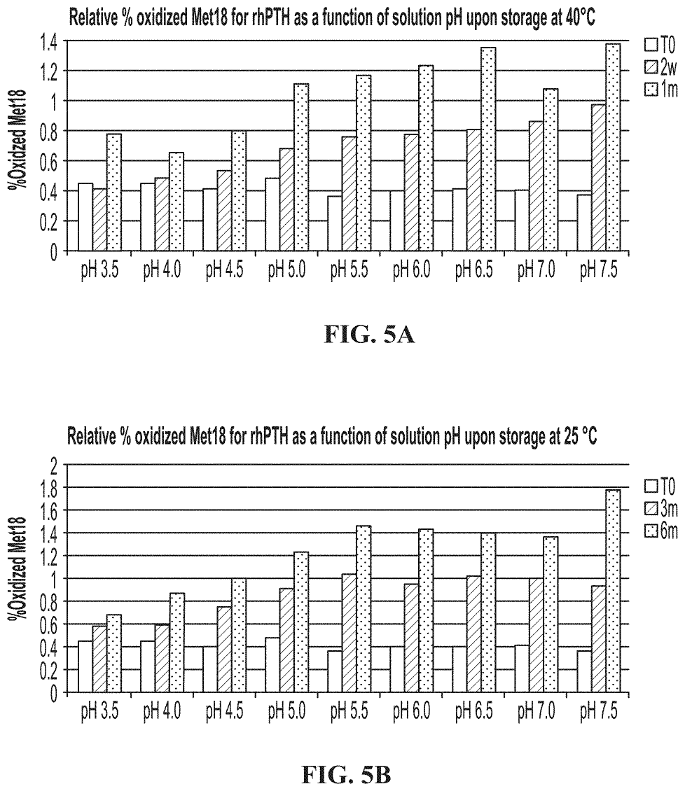

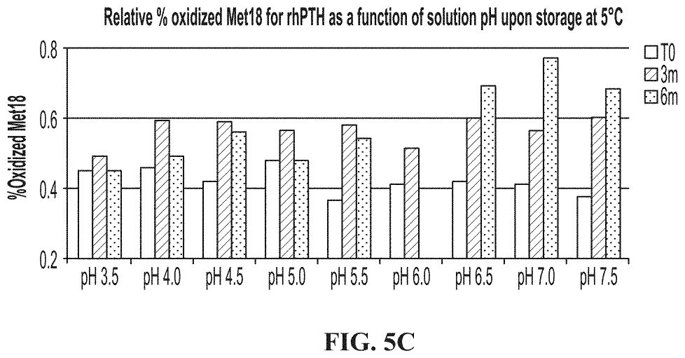

Oxidized Met18: Met18 oxidation rates were found to be maximum towards the basic solution pH range and decrease gradually as the solution pH became acidic. This trend was mainly visible at both 40 and 25° C. storage conditions, as shown in FIGS. 5A-5C.

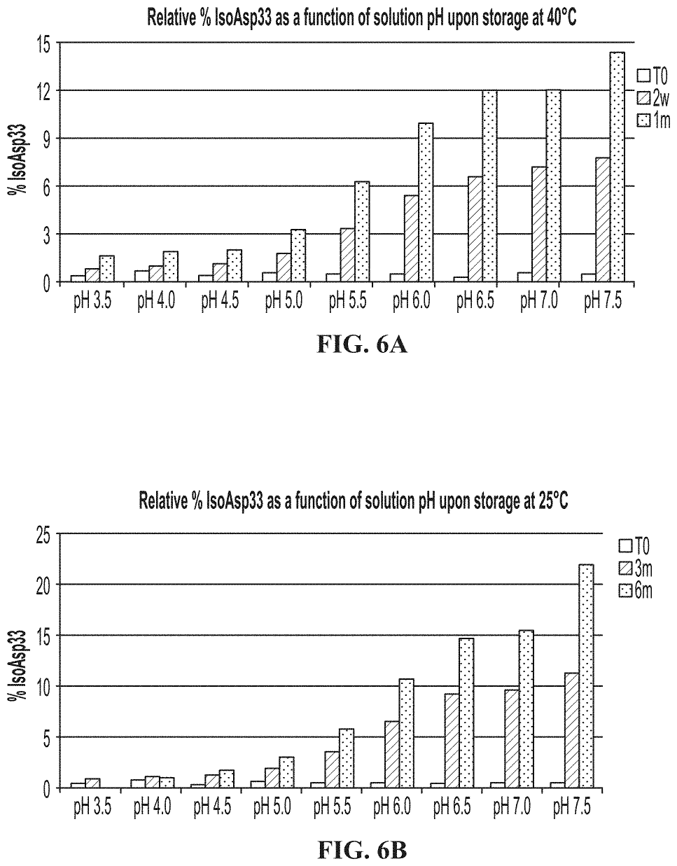

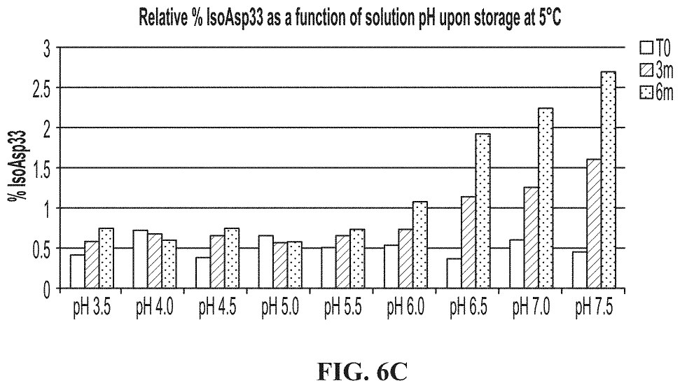

IsoAsp33: Formation of isoaspartate from asparagine33 was observed to be minimum towards the acidic side of formulated pH and was observed to increase significantly as the solution pH increases past ˜5.5. This trend was clear at all storage temperatures, as shown in FIGS. 6A-6C.

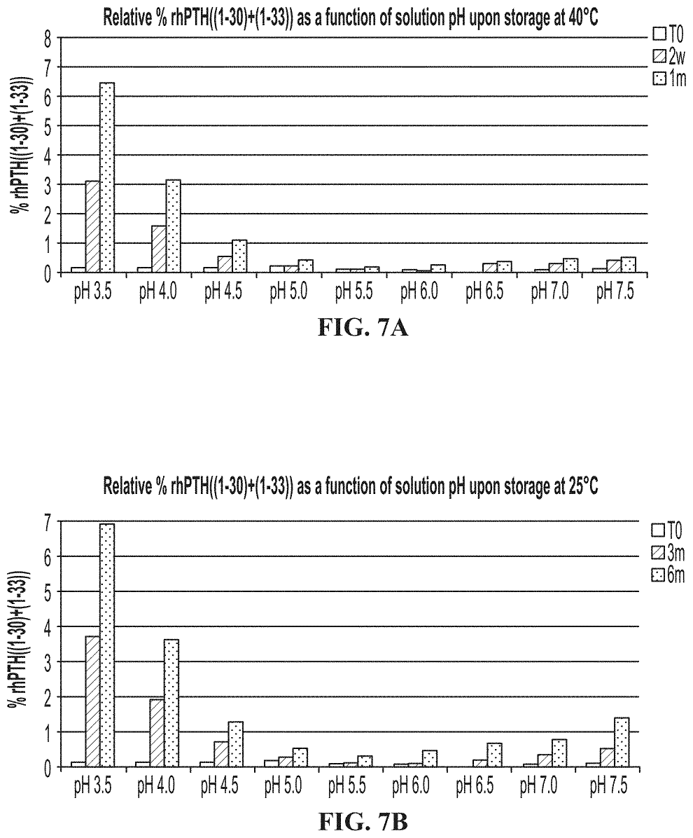

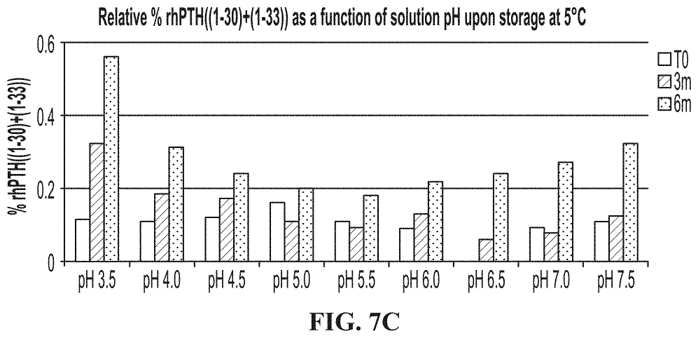

rhPTH((1-30)+(1-33)): These rhPTH impurities increased significantly upon storage in samples formulated below pH 5.0 and above pH 6.0. The increase in impurities above pH 6.0, however, occurred to a significantly lesser extent than that observed at lower pH values. This increase in impurity was observed to be minimum in the pH range of 5.0-6.0, as shown in FIGS. 7A-7C.

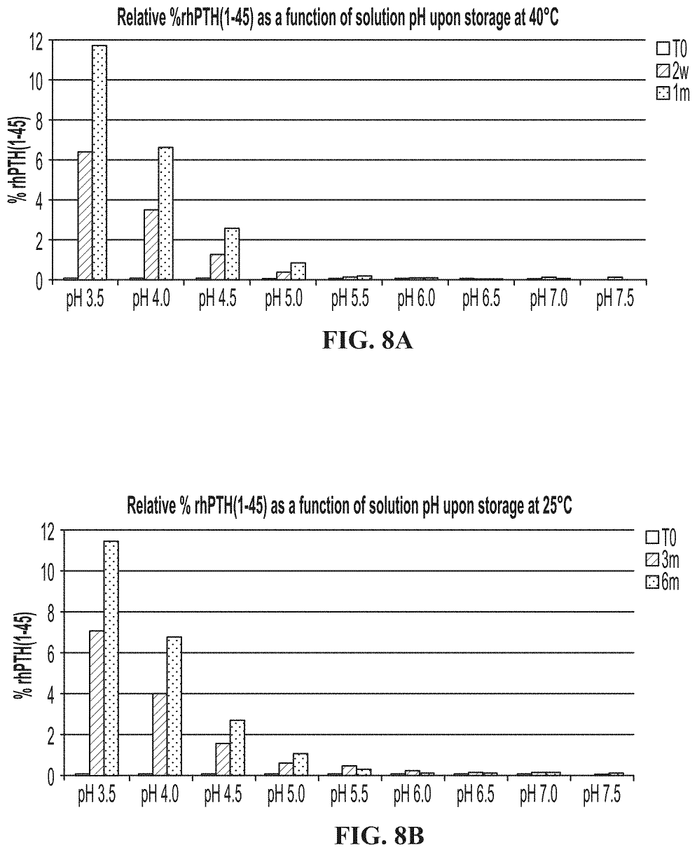

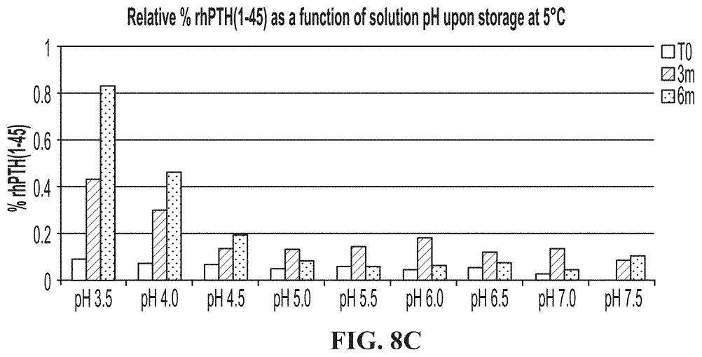

rhPTH(1-45): This fragment related impurity was observed to increase significantly in samples formulated below pH 5.0 and did not change significantly in samples between pH 5.0 and 7.5. This trend was seen at all storage temperatures, as shown in FIGS. 8A-8C.

The present example demonstrates the impact of solution pH on the physicochemical stability of rhPTH when formulated in a pH range of 3.5 to 7.5 and exposed to thermal stress. Physical stability attributes, as monitored using appearance (visible particle formation) and SEC (aggregates and fragments formation), and chemical stability attributes, as monitored using RP-HPLC (oxidation, deamidation, and fragmentation), suggest that a solution pH range of 5.0-6.0 is optimal for physical and chemical stability of rhPTH.

### Example 8: Excipient Screening for rhPTH for Optimal Physicochemical Stability

Recombinant human parathyroid hormone (rhPTH) was formulated in solution of pH 5.5 with 20 mM sodium acetate buffer along with 50 mM sodium chloride (NaCl). This base formulation was spiked with excipient stock to achieve desired target levels of a given excipient. The samples were placed on stability at temperature conditions of 5±3° C. (5° C.), 25±2° C. (25° C.), and 40±2° C. (40° C.). At pre-defined intervals, samples were pulled, observed for appearance, and analyzed for rhPTH stability using Reversed-Phase Chromatography (RP-HPLC). Baseline (time 0) samples were also exposed separately to multiple freeze-thaw cycles and orbital agitation and observed for solution appearance.

Appearance data from thermal stress over quiescent storage, freeze-thaw stress, and agitation stress showed that presence of arginine and higher levels (>150 mM) of NaCl resulted in significant levels of visible particle formation as compared to other excipients. RP-HPLC stability data showed significantly higher levels of oxidized Met8 and Met18 in samples containing glycine, lysine, or arginine at all incubation temperatures. On the other hand, samples with methionine showed significantly reduced rate of rhPTH oxidation. Results from agitation studies showed that the presence of surfactant, Poloxamer-188, prevented the formation of visible particles upon shaking.

The supplied drug substance material was thawed and dialyzed against the base buffer solution in 2 kDa MWCO dialysis cassette. The dialysis was performed at 5±3° C. and included at least 3 cycles of buffer exchanges over a period of −30 hours. Post dialysis, samples were tested for pH and adjusted if necessary with 0.2 N sodium hydroxide. A280 measurement was performed and rhPTH concentration was calculated based on an extinction coefficient of 0.584 (mL·mg)⁻¹cm⁻¹. Final solution preparation was done aseptically in a laminar flow hood. For each excipient, rhPTH was prepared at a concentration of 1.0 mg/mL by using the base buffer as the dilution medium and spiking the excipient stock to achieve the desired excipient concentration. Additionally, m-cresol was added at a level of 0.3% (v/v) in each of the formulations.

Table 10 provides the description of the different formulations used for the excipient screen study. The prepared samples were filtered via a 0.22 μm PVDF filter, filled at 1.5 mL volume in 2R Type I glass vials, followed by stoppering/crimping. Each vial was observed for solution appearance in a light box. Baseline samples were aliquoted in polypropylene tubes and stored at −80° C. Remaining vials were incubated at 5, 25, and 40° C. At predefined intervals, sample vials were pulled from each incubation condition, observed for appearance, aliquoted in polypropylene tubes, and stored at −80° C. until further analysis. Samples were tested for physical and chemical changes using assays validated for Natpara, including SEC and RP-HPLC with some modifications in injection volume and injection sequence.

Baseline samples were exposed to repetitive freeze/thaw cycles (freeze at −80° C. for 5-12 hours and thaw at room temperature) and observed for solution appearance in a lightbox. A different set of baseline samples in vials were agitated horizontally under ambient temperature conditions using an orbital shaker at 220 rpm and observed for solution appearance on regular intervals in a lightbox. Preliminary results from agitation studies were used to select additional formulations which were further exposed to orbital agitation in 2R vials and dual-chamber cartridges.

TABLE 10
Excipients (and concentrations) used in rhPTH
excipient screen study

| Base formulation | Excipients |
|---|---|
| rhPTH | NaC1 (50, 150, and 300 mM) |
| (1 mg/mL) + | Polysorbate 20 (0.005% and 0.02%) |
| Acetate | Poloxamer 188 (0.3% and 0.1%) |
| (20 mM) + | Sucrose (2% and 8%) |
| 50 mM NaCl | Glycerol (2% and 8%) |
| m-cresol + | Mannitol (50 and 150 mM) |
| (0.3%) | Glycine (50 and 150 mM) |
| | Methionine (50 and 100 mM |
| | Arginine (50 and 150 mM) |
| | Lysine (50 and 150 mM) |

Tables 11-13, below, shows the appearance results for the rhPTH stability samples stored at 40, 25, and 5° C., respectively, for up to 6 months. Opalescence against a reference suspension is noted where measured. Upon storage at 40° C., samples containing arginine (150 mM) showed a significant presence of proteinaceous particles which appeared at 2 weeks and kept on increasing over time. Samples with other excipients, when stored at 40° C., had appearance comparable to baseline for up to 3 months. By the end of 6 months storage at 40° C., most of the samples had visible particles present with varying color and opalescence. Similarly, for the samples stored at 25° C., arginine (150 mM) containing solution was the first one to show particle formation which did not appear until 6 months of storage, while all other samples maintained their baseline appearance. Samples stored at 5° C. had baseline like appearance at the end of 3 months storage, however, by the end of 6 months, many samples (specifically NaCl, glycerol, glycine, lysine, and arginine) had visible particles present unlike the results at 25° C.

TABLE 11
Appearance results over time for rhPTH
buffer screen samples stored at 40° C.

| | 40° C. | | | |
|---|---|---|---|---|
| Excipient | T 0 | 1 m | 3 m | 6 m |
| Control (no m-cresol) | CCFVP | CCFVP | CCFVP | CCFVP |
| NaCl (50 mM) | CCFVP | CCFVP | CCFVP | CCFVP |
| NaCl (150 mM) | CCFVP | CCFVP | CCFVP | White, fibrous |
| | | | | proteinaceous |
| | | | | particles (++) |
| NaCl (300 mM) | CCFVP | CCFVP | CCFVP | White, fibrous |
| | | | | proteinaceous |
| | | | | particles (++) |
| PS20 (0.005%) | RS I | CCFVP | CCFVP | CCFVP |
| | (no particles) | | | |
| PS20 (0.02%) | RS II and | RS II and | RS II and | RS II |
| | RS III (granular | RS III (granular | RS III (granular | (granular |
| | non-proteinaceous | non-proteinaceous | non-proteinaceous | non-proteinaceous |
| | particles) | particles) | particles) | particles) |
| P-188 (0.3%) | CCFVP | RS I (no particles) | W and | CCFVP |
| | | | RS I (no particles) | |
| P-188 (1%) | CCFVP | RS II and RS III | RS I and RS II | RS I |
| | | (no particles) | (no particles) | (no particles) |
| Sucrose (2%) | CCFVP | CCFVP | CCFVP | CCFVP |
| Sucrose (8%) | CCFVP | CCFVP | CCFVP | White fiber |
| | | | | like particles (+) |
| Glycerol (2%) | CCFVP | CCFVP | CCFVP | W and RS I (white |
| | | | | fiber like particles |
| | | | | (++)) |
| Glycerol (8%) | CCFVP | CCFVP | CCFVP | W and RS I (white |
| | | | | fiber like particles |
| | | | | (+)) |
| Mannitol (50 mM) | CCFVP | CCFVP | CCFVP | RS I (white |
| | | | | fiber like particles |
| | | | | ++)) |
| Mannitol (150 mM) | CCFVP | CCFVP | CCFVP | RS I (white |
| | | | | fiber like particles |
| | | | | +)) |
| Glycine (50 mM) | CCFVP | CCFVP | CCFVP | Yellowish tint, |
| | | | | few fibrous |
| | | | | particles (++) |
| Glycine (150 mM) | CCFVP | CCFVP | CCFVP | Yellowish tint, |
| | | | | few fibrous |
| | | | | particles (++) |
| Methionine (50 mM) | CCFVP | CCFVP | CCFVP | RS I and RS II |
| | | | | (particles ++) |
| Methionine | CCFVP | CCFVP | RS I and RS II | RS I and RS II |
| (100 mM) | | | (no particles) | (particles ++) |
| Lysine (50 mM) | CCFVP | CCFVP | CCFVP | Yellowish tint, |
| | | | Possible signs | few flocculants |
| | | | of particles | and fibrous |
| | | | | particles (++) |
| Lysine (150 mM) | CCFVP | CCFVP | CCFVP | Yellowish tint, |
| | | | | few flocculants |
| | | | | and fibrous |
| | | | | particles (++) |
| Arginine (50 mM) | CCFVP | CCFVP | CCFVP | Few particles (+) |
| Arginine (150 mM) | CCFVP | RS II and | RS II and | RS II and |
| | | RS III (white | RS III (white | RS III (white |
| | | proteinaceous | proteinaceous | proteinaceous |
| | | flocculants | flocculants | flocculants |
| | | ++) | +++) | +++) |

RS: Reference Suspension;
W: Water-like appearance;
CCFVP: Clear, colorless, free of visible particles

TABLE 12
Appearance results over time for rhPTH buffer
screen samples stored at 25° C.

| | 25° C. | | | |
|---|---|---|---|---|
| Excipient | T 0 | 1 m | 3 m | 6 m |
| Control (no m-cresol) | CCFVP | CCFVP | CCFVP | CCFVP |
| NaCl (50 mM) | CCFVP | CCFVP | CCFVP | CCFVP |
| NaCl (150 mM) | CCFVP | CCFVP | CCFVP | CCFVP |
| NaCl (300 mM) | CCFVP | CCFVP | CCFVP | CCFVP |
| PS20 (0.005%) | RS I | CCFVP | CCFVP | CCFVP |
| | (no particles) | | | |
| PS20 (0.02%) | RS II and | RS II and | RS II and | RS II |
| | RS III (granular | RS III (granular | RS III (granular | (granular |
| | non-proteinaceous | non-proteinaceous | non-proteinaceous | non-proteinaceous |
| | particles visible) | particles visible) | particles visible) | particles visible) |
| P-188 (0.3%) | CCFVP | CCFVP | CCFVP | CCFVP |
| P-188 (1%) | CCFVP | RS I and RS II | RS I and RS II | RS II |
| | | (no particles) | (no particles) | (no particles) |
| Sucrose (2%) | CCFVP | CCFVP | CCFVP | RS I |
| | | | | (no particles) |
| Sucrose (8%) | CCFVP | CCFVP | CCFVP | CCFVP |
| Glycerol (2%) | CCFVP | CCFVP | CCFVP | CCFVP |
| Glycerol (8%) | CCFVP | CCFVP | CCFVP | CCFVP |
| Mannitol (50 mM) | CCFVP | CCFVP | CCFVP | CCFVP |
| Mannitol (150 mM) | CCFVP | CCFVP | CCFVP | CCFVP |
| Glycine (50 mM) | CCFVP | CCFVP | CCFVP | RS I |
| | | | | (no particles) |
| Glycine (150 mM) | CCFVP | CCFVP | CCFVP | RS I |
| | | | | (no particles) |
| Methionine (50 mM) | CCFVP | CCFVP | CCFVP | RS I |
| | | | | (no particles) |
| Methionine (100 mM) | CCFVP | CCFVP | CCFVP | CCFVP |
| Lysine (50 mM) | CCFVP | CCFVP | CCFVP | RS I |
| | | | | (no particles +) |
| Lysine (150 mM) | CCFVP | CCFVP | CCFVP | CCFVP |
| Arginine (50 mM) | CCFVP | CCFVP | CCFVP | RS I |
| | | | | (no particles) |
| Arginine (150 mM) | CCFVP | W and RS I | W and RS I | RS III |
| | | (no particles) | (no particles) | (white |
| | | | | proteinaceous |
| | | | | flocculants +++) |

RS: Reference Suspension;
W: Water-like appearance;
CCFVP: Clear, colorless, free of visible particles

TABLE 13
Appearance results over time for rhPTH buffer
screen samples stored at 5° C.

| | 5° C. | | | |
|---|---|---|---|---|
| Excipient | T 0 | 1 m | 3 m | 6 m |
| Control (no m-cresol) | CCFVP | CCFVP | CCFVP | CCFVP |
| NaCl (50 mM) | CCFVP | CCFVP | CCFVP | RS I |
| | | | | (with |
| | | | | proteinaceous |
| | | | | particles +++) |
| NaCl (150 mM) | CCFVP | CCFVP | CCFVP | W (with |
| | | | | proteinaceous |
| | | | | particles ++) |
| NaCl (300 mM) | CCFVP | CCFVP | CCFVP | RSI |
| | | | | (with |
| | | | | proteinaceous |
| | | | | particles +++) |
| PS20 (0.005%) | RS I | CCFVP | CCFVP | CCFVP |
| | (no particles) | | | |
| PS20 (0.02%) | RS II and | RS I | RS II and | RS III |
| | RS III (granular | (granular | RS III (granular | (granular |
| | non-proteinaceous | non-proteinaceous | non-proteinaceous | non-proteinaceous |
| | particles visible) | particles visible) | particles visible) | particles visible) |
| P-188 (0.3%) | CCFVP | CCFVP | CCFVP | CCFVP |
| P-188 (1%) | CCFVP | CCFVP | CCFVP | RS I |
| | | | | (no particles) |
| Sucrose (2%) | CCFVP | CCFVP | CCFVP | RS I |
| | | | | (no particles) |
| Sucrose (8%) | CCFVP | CCFVP | CCFVP | W (possible |
| | | | | signs of particles) |
| Glycerol (2%) | CCFVP | CCFVP | CCFVP | RS I |
| | | | | (no particles) |
| Glycerol (8%) | CCFVP | CCFVP | CCFVP | RS III (very fine |
| | | | | particles +) |
| Mannitol (50 mM) | CCFVP | CCFVP | CCFVP | RS I |
| | | | | (no particles) |
| Mannitol (150 mM) | CCFVP | CCFVP | CCFVP | CCFVP |
| Glycine (50 mM) | CCFVP | CCFVP | CCFVP | CCFVP |
| Glycine (150 mM) | CCFVP | CCFVP | CCFVP | RS II (very fine |
| | | | | particles ++) |
| Methionine (50 mM) | CCFVP | CCFVP | CCFVP | CCFVP |
| Methionine (100 mM) | CCFVP | CCFVP | CCFVP | CCFVP |
| Lysine (50 mM) | CCFVP | CCFVP | CCFVP | CCFVP |
| Lysine (150 mM) | CCFVP | CCFVP | CCFVP | RS I (very few |
| | | | | particles +) |
| Arginine (50 mM) | CCFVP | CCFVP | CCFVP | CCFVP |
| Arginine (150 mM) | CCFVP | CCFVP | CCFVP | RS III |
| | | | | (white |
| | | | | proteinaceous |
| | | | | flocculants ++) |

RS: Reference Suspension;
W: Water-like appearance;
CCFVP: Clear, colorless, free of visible particles

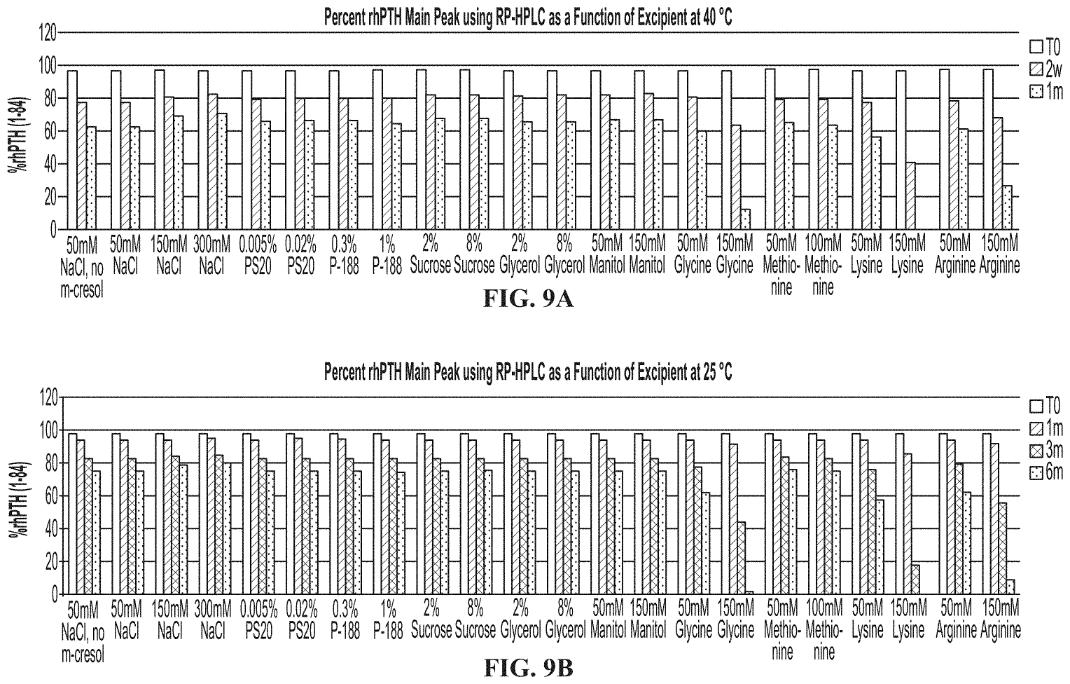

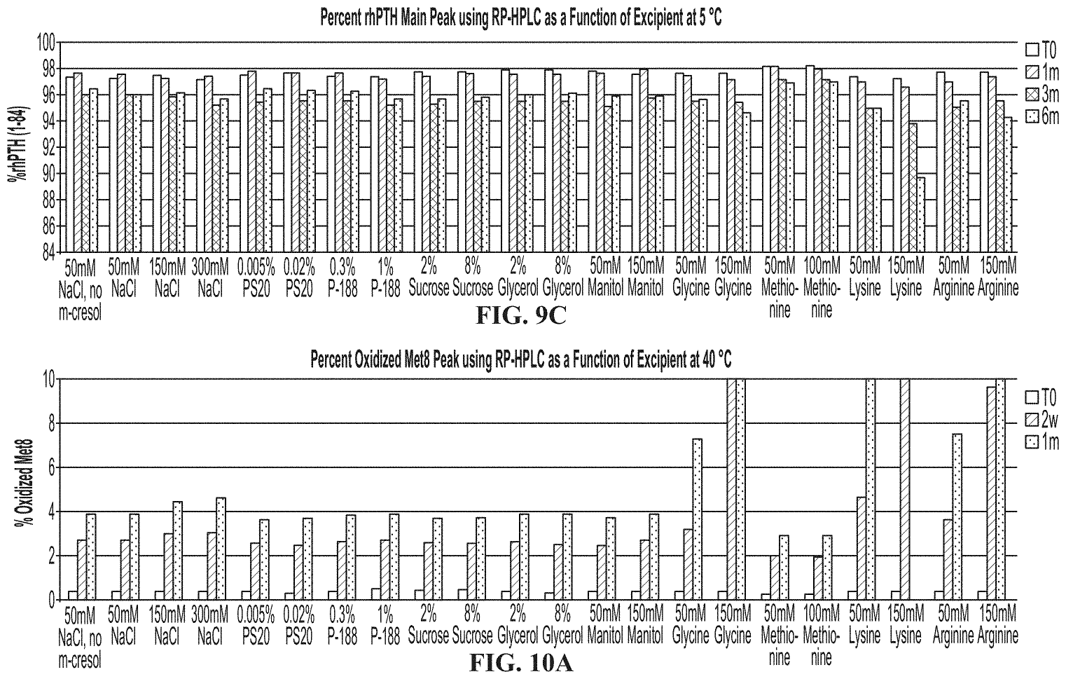

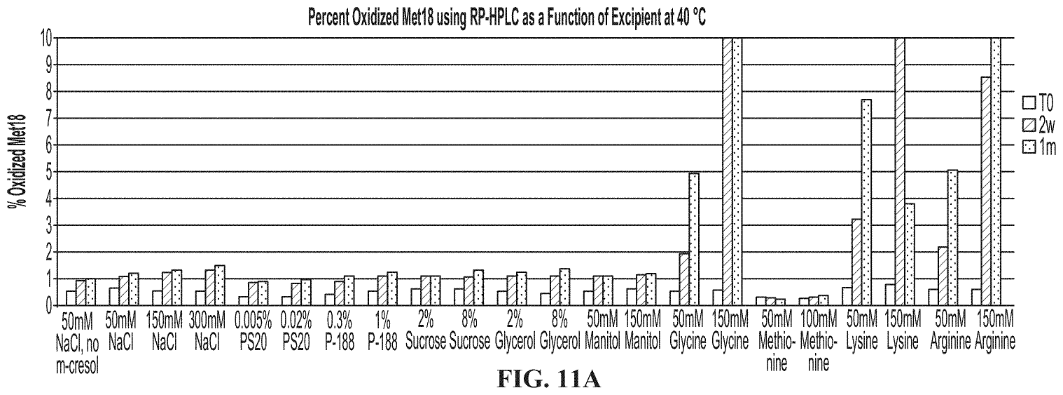

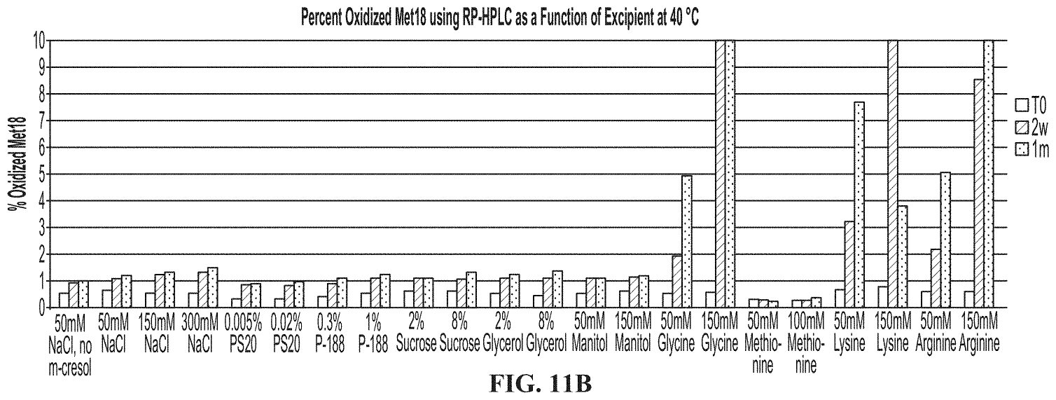

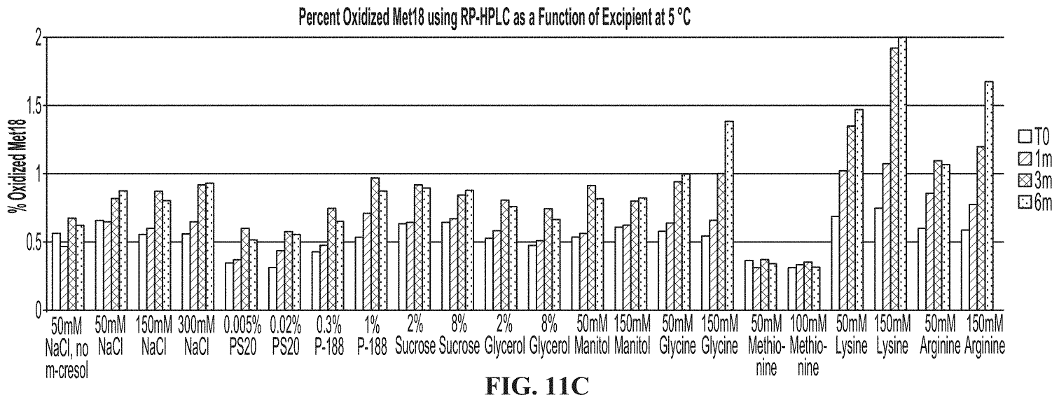

To demonstrate chemical stability, FIGS. 9-12 show the RP-HPLC data for rhPTH and associated impurities for samples formulated in pH 5.5 acetate buffer containing 50 mM NaCl with different excipients and stored at 40, 25, and 5° C. Results are presented for the samples where reasonable peak integration was possible without a shift in the reported relative retention times.

Main peak: Samples containing glycine, lysine, and arginine showed a significantly faster decrease of the main peak compared to other excipients at both 40 and 25° C. storage. Similar trend was observed at 5° C. storage, see FIGS. 9A-9C.

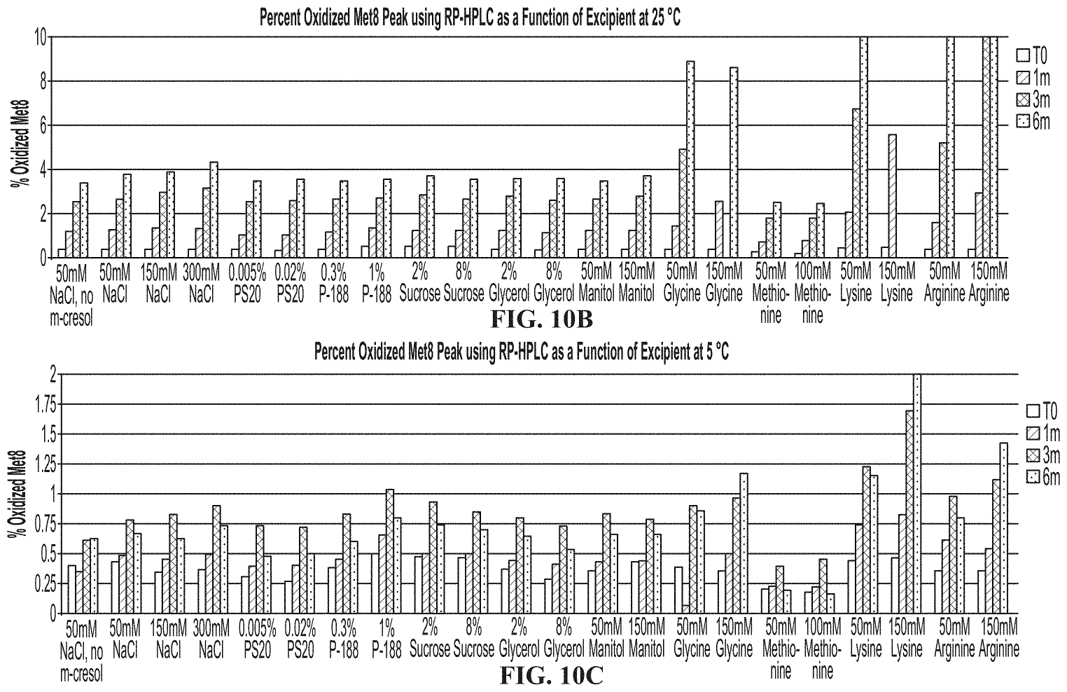

Oxidized Met8 and Met18: Compared to other excipients, glycine, lysine, and arginine samples showed significantly higher levels of oxidized Met8 and Met18 at all storage temperatures. Samples containing methionine showed the least change in the Met8 and Met18 oxidation over time at all storage temperatures, see FIGS. 10A-10C (Met8) and 11A-11C (Met18).

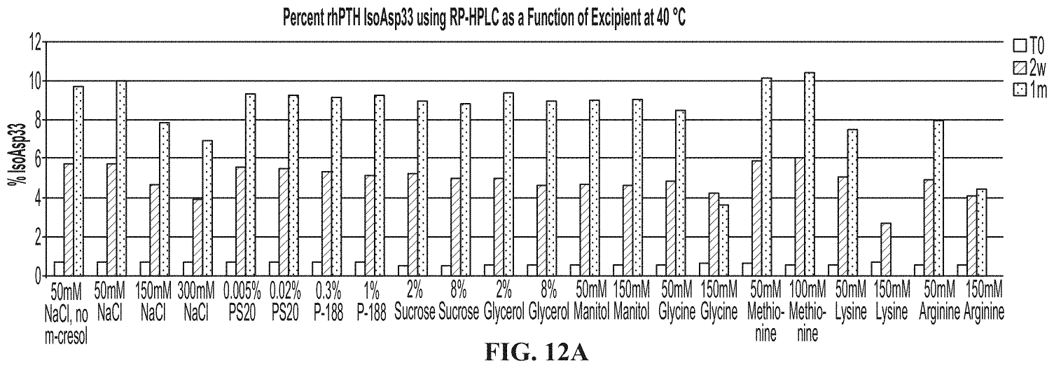

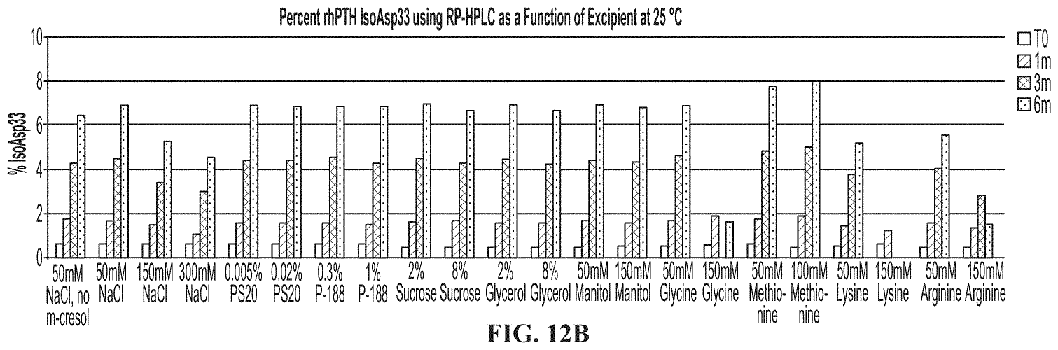

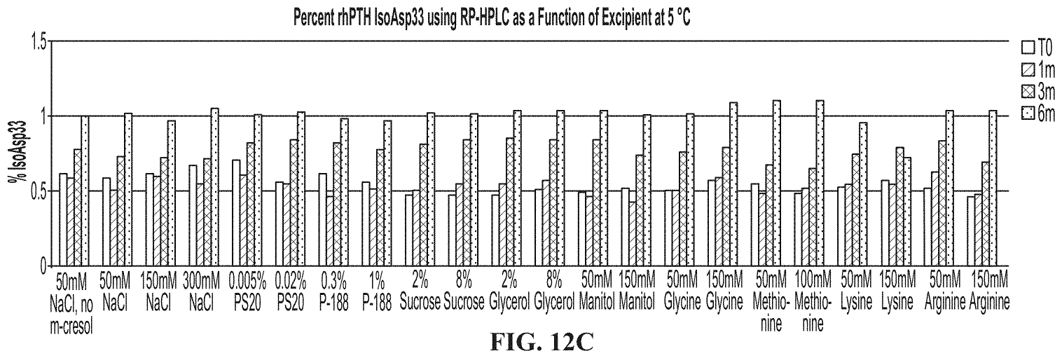

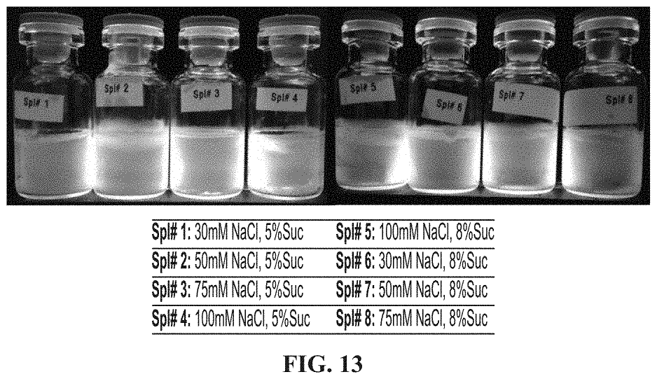

IsoAsp33: Upon 25 and 40° C. storage, although samples with 150 and 300 mM NaCl showed slightly lower rate of IsoAsp33 formation, no significant differences among the excipients were noticeable (significantly lower and inconsistent IsoAsp33 levels observed with glycine, lysine, and arginine samples can be attributed to the issues in integrating missing peak/peak with slightly shifted retention time in chromatograms of these samples), see FIGS. 12A-12C.

Freeze-Thaw (F/T) Studies

Table 14 shows the solution appearance of different formulations upon repetitive freeze-thaw performed in 2R vials. Visible particles, if observed, are reported alongside the opalescence. Samples with 150 mM NaCl or higher were significantly impacted by repetitive F/T and were found to contain white, fibrous protein-like particles. Samples with 0.02% PS20 showed granular appearance because of sand-like (non-proteinaceous) particles from the beginning. Solutions with 8% glycerol showed worsening opalescence after each F/T cycle without any visible particulate formation.

TABLE 14
Appearance study results for rhPTH samples formulated with different
excipients at pH 5.5 with 20 mM acetate buffer, 50 mM NaCl, and 0.3%
m-cresol upon repetitive freeze-thaw cycles (n =3)

| Formulation* | Baseline | 1 F/T | 2 F/T | 3 F/T |
|---|---|---|---|---|
| 50 mM NaCl, | CCFVP | CCFVP | CCFVP | CCFVP |
| no m-cresol | | | | |
| 50 mM NaCl | CCFVP | CCFVP | CCFVP | CCFVP |
| 150 mM NaCl | CCFVP | RS II | RS III | RS III |
| | | | | (white, fibrous |
| | | | | proteinaceous |
| | | | | particles) |
| 300 mM NaCl | CCFVP | RS III | RS IV and | RS IV and |
| | | | SOP | SOP |
| | | | (white, fibrous | (white, fibrous |
| | | | proteinaceous | proteinaceous |
| | | | particles) | particles) |
| 0.005% PS20 | RS I | RS I | RS I | RS I and II |
| 0.02% PS20 | RS III | RS III | Between | RS III |
| | (granular | (granular | RS III | (granular |
| | sandy particles, | sandy particles, | and RS IV | sandy particles, |
| | non- | non- | (granular | non- |
| | proteinaceous) | proteinaceous) | sandy particles, | proteinaceous) |
| | | | non- | |
| | | | proteinaceous) | |
| 0.3% P-188 | CCFVP | CCFVP | W and RS I | CCFVP |
| 1% P-188 | CCFVP | RS II | CCFVP | CCFVP |
| 2% Sucrose | CCFVP | CCFVP | RS I | RS I |
| 8% Sucrose | CCFVP | RS I | RS III | Between |
| | | | | RS II and RS III |
| 2% Glycerol | CCFVP | RS I | Between | RS II |
| | | | RS I and RS II | |
| 8% Glycerol | CCFVP | RS II | RS II | RS III |
| 50 mM Mannitol | CCFVP | RS I | RS I | RS I |
| 150 mM Mannitol | CCFVP | RS II | RS I | RS I |
| 50 mM Glycerol | CCFVP | RS I | RS I | RS I |
| 150 mM Glycerol | CCFVP | RS II | RS II | RS I |
| 50 mM Methionine | CCFVP | RS I | RS I | RS I |
| 100 mM Methionine | CCFVP | CCFVP | RS I | RS I |

*Excipients were spliced into a base formulation containing 1 mg/mL rhPTH, 20 mM acetate buffer, 50 mM sodium chloride, and 0.3% m-cresol.
CCFVP: Clear, colorless, and free of visible particles;
RS: Reference Suspension;
SOP: Standard of opalescence;
W: Water-like appearance.
Lysine and Arginine samples could not be studied due to material limitations.

Agitation Studies

Table 15 shows the appearance results from the agitation studies performed on triplicate 2R vials in horizontal position at 220 rpm under ambient conditions. All samples were clear, colorless, and free of visible particles at baseline, except PS20, which had sand-like particles present. Samples with NaCl showed the earliest sign of particle formation, the rate of which increased with increasing NaCl content. By 24 hours, samples with 150 mM NaCl and PS20 had turbid appearance. All samples except the ones with Poloxamer-188 (P-188) developed turbid appearance by the end of 48 hours. Samples with P-188 maintained their baseline appearance until the end of study (72 hours).

TABLE 15
Appearance study results for rhPTH samples formulated with different excipients at pH 5.5 with 20 mM acetate buffer, 50 mM NaCl, and
0.3% m-cresol upon orbital agitation at 220 rpm under ambient conditions in 2R vials (n = 3)

| Formulation* | T0 | 4 h | 8 h | 24 h | 48 h | 72 h |
|---|---|---|---|---|---|---|
| 50 mM NaCl, no | CCFVP | CCFVP | CCFVP | CCFVP | Between RS II | Between RS IV |
| m-cresol | | | | | and RS III | and SOP |
| 50 mM NaCl | CCFVP | CCFVP | CCFVP | Between RS III and RS IV | >SOP (turbid) | >SOP (turbid) |
| 150 mM NaCl | CCFVP | CCFVP | CCFVP | >SOP (turbid) | >SOP (turbid) | >SOP (turbid) |
| 300 mM NaCl | CCFVP | RS III | >SOP | >SOP (turbid) | >SOP (turbid) | >SOP (turbid) |
| 0.005% PS20 | CCFVP | Between W | RS I | >SOP | >SOP (turbid) | >SOP (turbid) |
| | | and RS I | | | | |
| 0.02% PS20 | RS I (granular | RS IV | Between RS IV and SOP | >SOP | >SOP | >SOP (turbid) |
| | sandy particles, | | | (turbid) | (turbid) | |
| | non-proteinaceous) | | | | | |
| 0.3% P-188 | CCFVP | CCFVP | Between W and RS I | Between W and RS I | CCFVP | Between W |
| | | | | | | and RS I |
| 1% P-188 | CCFVP | RS II | Between RS I and RS II | Between RS I and RS II | Between RS I | Between RS I |
| | | | | | and RS II | and RS II |
| 2% Sucrose | CCFVP | CCFVP | CCFVP | Between RS II and RS III | >SOP (turbid) | >SOP (turbid) |
| 8% Sucrose | CCFVP | CCFVP | CCFVP | Between RS IV and SOP | >SOP (turbid) | >SOP (turbid) |
| 2% Glycerol | CCFVP | CCFVP | Between W and RS I | Between RS II and RS III | >SOP (turbid) | >SOP (turbid) |
| 8% Glycerol | CCFVP | CCFVP | CCFVP | Between RS IV and SOP | >SOP (turbid) | >SOP (turbid) |
| 50 mM Mannitol | CCFVP | CCFVP | CCFVP | Between W and RS I | >SOP (turbid) | >SOP (turbid) |
| 150 mM Mannitol | CCFVP | CCFVP | CCFVP | Between RS I and RS II | >SOP (turbid) | >SOP (turbid) |
| 50 mM Glycine | CCFVP | CCFVP | CCFVP | Between RS III and RS IV | >SOP (turbid) | >SOP (turbid) |
| 150 mM Glycine | CCFVP | CCFVP | CCFVP | RS IV | >SOP (turbid) | >SOP (turbid) |
| 50 mM Methionine | CCFVP | CCFVP | CCFVP | Between RS I and RS II | >SOP (turbid) | >SOP (turbid) |
| 100 mM | CCFVP | CCFVP | CCFVP | Between RS II and RS III | >SOP (turbid) | >SOP (turbid) |
| Methionine | | | | | | |

*Excipients were spiked into a base formulation containing 1 mg/mL rhPTHm 20 mM acetate buffer, 50 mM sodium chloride, and 0.3% m-cresol. CCFVP: Clear, colorless, and free of visible particles; RS: Reference suspension; SOP: Standard of opalescence; W: Water-like appearance. Lysine and Arginine samples could not be studied due to material limitations.

Based on the preliminary results from storage stability, freeze-thaw, and agitation studies above, formulations were narrowed down where NaCl, mannitol, sucrose, and glycerol were identified as excipients for their stabilization/isotonicity potential, along with methionine and m-cresol to mitigate oxidation and to support a multi-dose formulation, respectively.

Table 16 presents the results from horizontal agitation studies performed at 220 rpm under ambient conditions in 2R vials where NaCl was removed from the base formulation. Presence of m-cresol resulted in opalescence formation significantly earlier than non m-cresol formulations. Despite the removal of 50 mM NaCl from the base formulation, all the solutions still resulted in a turbid appearance by the end of 48 hours shaking except the ones with Poloxamer-188. These formulations were also exposed to shaking in the container/closure currently used for commercial Natpara® (1 mL siliconized cartridges with siliconized middle and end rubber stoppers and aluminum seal using a 1.1 mL formulation fill). Appearance results similar to that obtained with shaking in 2R vials (Table 15) were observed, where Poloxamer-188 significantly prevented/delayed the particulate formation.

TABLE 16
Appearance study results for rhPTH samples formulated with narrowed down excipients at pH 5.5 with 20 mM acetate buffer,
with and without methionine and m-cresol, upon orbital agitation at 220 rpm under ambient conditions in 2R vials (n = 3).

| Formulations* | T0 | 4 h | 8 h | 24 h | 48 h | 58 h |
|---|---|---|---|---|---|---|
| Control | CCFVP | CCFVP | CCFVP | CCFVP | Between RS III and RS IV | Between RS III and RS IV |
| (20 mM Acetate) | | | | | | |
| Control + Meth + | CCFVP | CCFVP | CCFVP | Between W and RS I | >SOP (turbid) | >SOP (turbid) |
| m-cresol | | | | | | |
| Control + m-cresol | CCFVP | CCFVP | CCFVP | Between RS I and RS II | >SOP (turbid) | >SOP (turbid) |
| Control + Met | CCFVP | CCFVP | CCFVP | CCFVP | Between RS II and RS IV | Between RS III and RS IV |
| 50 mM NaCl | CCFVP | CCFVP | CCFVP | CCFVP | Between RS III and RS IV | Between RS III and RS IV |
| 50 mM NaCl + | CCFVP | CCFVP | CCFVP | Between W and RS I | >SOP (turbid) | >SOP (turbid) |
| Met + m-cresol | | | | | | |
| 5% Mannitol | CCFVP | CCFVP | CCFVP | CCFVP | Between RS III and RS IV | Between RS III and RS IV |
| 5% Mannitol + | CCFVP | CCFVP | CCFVP | Between RS II and | >SOP (turbid) | >SOP (turbid) |
| Met + m-cresol | | | | RS III | | |
| 5% Sucrose | CCFVP | CCFVP | CCFVP | CCFVP | Between RS III and RS IV | Between RS III and RS IV |
| 5% Sucrose + | CCFVP | CCFVP | CCFVP | Between RS I and RS II | >SOP (turbid) | >SOP (turbid) |
| Met + m-cresol | | | | | | |
| 2% Glycerol | CCFVP | CCFVP | CCFVP | Between W and RS I | >SOP (turbid) | >SOP (turbid) |
| 2% Glycerol + | CCFVP | CCFVP | CCFVP | Between W and RS I | >SOP | >SOP (turbid) |
| Met + m-cresol | | | | | | |
| 0.3% P-188 | CCFVP | CCFVP | CCFVP | CCFVP | CCFVP | CCFVP |
| 0.3% P-188 + | CCFVP | Between W | Between W | Between W and RS I | Between W andRS I | Between W and RS I |
| Met + m-cresol | | and RS I | and RS I | | | |

*Excipients were spiked into a base formulation containing 1 mg/mL rhPTH and 20 mM acetate buffer. Met: Methionine (100 mM); m-cresol (0.3%); CCFVP: Clear, colorless, and free of visible particles; RS: Reference suspension; SOP: Standard of opalescence; W: Water-like appearance.

All the formulations studied in Table 16 maintained their baseline (clear) appearance at the end of 72 hours period (2R vials) or 48 hours period (1 mL cartridges) when shaking was performed at 2-8° C.

As the example demonstrates, sodium chloride, sucrose, mannitol, and glycerol are suitable excipients to provide stabilization to rhPTH. Methionine exhibits high potential to significantly inhibit peptide oxidation. Poloxamer-188 has been found to be critical to prevent visible particulate formation upon agitation.

### Example 9: Formulation Optimization Studies for rhPTH Targeting a Liquid Dosage Form

Recombinant human parathyroid hormone was formulated in pH 5.5 with 20 mM acetate buffer and 0.3% w/v m-cresol. This base formulation was prepared with varying levels of methionine (antioxidant) and Poloxamer-188 (surfactant), excipients identified critical for rhPTH stability during early formulation screening (see Example 7). To make the formulation isotonic and further improve the stability of drug product, sodium chloride, sucrose, glycerol, and mannitol were evaluated. To screen excipients and optimize their concentrations, samples were exposed to thermal and agitation stresses. For thermal stress, samples were placed on stability in 2R Type I glass vials at 5±3° C. (5° C.), 25±2° C. (25° C.), and 40±2° C. (40° C.) and at pre-defined intervals, samples were pulled, observed for appearance, and analyzed for rhPTH stability using Reversed-Phase Chromatography (RP-HPLC). For agitation stresses, baseline samples in 2R Type I glass vials and 1 mL siliconized dual-chamber cartridges were exposed separately to orbital agitation and observed for solution appearance over time.

No differences between the oxidation profile of rhPTH were observed between 50 mM, 25 mM and 10 mM methionine containing formulations upon thermal stress. Visible particle formation in rhPTH solution upon agitation was not observed to be dependent on the concentration of Poloxamer-188. Stabilizers/tonicity agents, in the form of NaCl, sucrose, and glycerol, were selected from a combination of chemical and physical changes in the molecule observed upon thermal and agitation stresses performed during the preliminary excipient screen (see Example 7). The concentration of stabilizers/tonicity agents were selected to achieve isotonic solutions. Overall, three formulation matrices targeting a liquid dosage form were identified:

a) pH 5.5, 20 mM Acetate Buffer, 10 mM Methionine, 0.3% w/v Poloxamer-188, 130 mM Sodium Chloride, 0.3% w/v m-cresol

b) pH 5.5, 20 mM Acetate Buffer, 10 mM Methionine, 0.3% w/v Poloxamer-188, 8.5% w/v Sucrose, 0.3% w/v m-cresol

c) pH 5.5, 20 mM Acetate Buffer, 10 mM Methionine, 0.3% w/v Poloxamer-188, 2.3% v/v Glycerol, 0.3% w/v m-cresol

The supplied drug substance material was thawed and dialyzed against the buffer solution in 2 kDa MWCO dialysis cassette. The dialysis was performed at 5±3° C. and included at least 3 cycles of buffer exchanges over a period of −24-48 hours. Post dialysis, samples were tested for pH and pH was adjusted, if necessary, with 0.2 N sodium hydroxide. A280 measurement was performed and rhPTH concentration was calculated based on an extinction coefficient of 0.584 (mL·mg)⁻¹cm⁻¹. Final solution preparation was done aseptically in a laminar flow hood. rhPTH solution was prepared at a concentration of 1.0 mg/mL by using the base buffer as the dilution medium and spiking the excipient stocks to achieve the desired excipient concentration. Additionally, m-cresol was added at a level of 0.3% w/v in each of the formulations.

Methionine and Poloxamer-188 concentration optimization: Table 17 provides the description of different formulations used for methionine and P-188 concentration optimization studies.

TABLE 17
Formulations used in methionine and Poloxamer-188
(P-188) concentration optimization

| | P-188 concentration (%) with each | | | |
|---|---|---|---|---|
| Base | methionine (Met) sample | | | |
| formulation | 10 mM Met | 25 mM Met | 50 mM Met | |
| 1 mg/mL rhPTH | 0 | 0 | 0 | 0 |
| in 20 mM | 0.1 | 0.1 | 0.1 | 0.3 |
| acetate buffer | 0.2 | 0.2 | 0.2 | 0.5 |
| and 0.3% | 0.3 | 0.3 | 0.3 | 0.75 |
| m-cresol | 0.5 | 0.5 | 0.5 | 1.0 |

Optimization of stabilizer/tonicity agents: Table 18 provides the description of different formulations used for evaluating the impact of stabilizer/tonicity agents on rhPTH stability.

TABLE 18
Formulations used in the optimization of stabilizer/tonicity agent

| Base Formulation | Excipient |
|---|---|
| 1 mg/mLrhPTH in 20 mM acetate buffer, | None (Control) |
| 25 mM Methionine, 0.3% P-188, and | 130 mM NaC1 |
| 0.3% m-cresol | 8.5% w/v Sucrose |
| | 2.3% v/v Glycerol |
| | 250 mM Mannitol |

The samples were filtered via 0.22 μm PVDF filter, filled at a 1.5 mL volume in 2R Type I glass vials (for agitation) or at a 1 mL volume in 2R Type I glass vials (for storage stability), followed by stoppering/crimping. Each vial was observed for solution appearance in a light box. All the baseline samples in Table 17 and Table 18 were exposed to horizontal agitation under ambient temperature conditions using an orbital shaker at 220 rpm and observed for solution appearance on regular intervals in a lightbox.

Samples from Table 17, containing 0.3% Poloxamer-188 with 0 mM, 10 mM, 25 mM, and 50 mM methionine, and Table 18 were also placed on storage stability. Baseline samples were separated, aliquoted in polypropylene tubes, and stored at −80° C. Remaining vials were incubated at 5, 25, and 40° C. At predefined intervals, sample vials were pulled from each incubation condition, observed for appearance, aliquoted in polypropylene tubes, and stored at −80° C. until analysis. Samples were tested for physical and chemical changes using assays validated for Natpara, including SEC and RP-HPLC with some modifications in injection volume and injection sequence.

Appearance: Table 19 shows the appearance results for the rhPTH stability samples with different levels of methionine concentrations when stored at 40, 25, and 5° C. for up to 6 months. All samples stayed clear, colorless, and free of visible particles over the studied duration.

TABLE 19
Appearance results for rhPTH stability samples with different methionine concentrations
under different temperature conditions

| Formulation | | | | | | | | | |
|---|---|---|---|---|---|---|---|---|---|
| 1 mg/mL rhPTH in 20 mM | | | | | | | | | |
| acetate buffer, 0.3% | | 40° C. | | | 25° C. | | | 5° C. | |
| P-188, and 0.3% m-cresol with | T0 | 1 m | 3 m | 6 m | 1 m | 3 m | 6 m | 3 m | 6 m |
| 10 mM Met | CCFVP | CCFVP | CCFVP | CCFVP | CCFVP | CCFVP | CCFVP | CCFVP | CCFVP |
| 25 mM Met | CCFVP | CCFVP | CCFVP | CCFVP | CCFVP | CCFVP | CCFVP | CCFVP | CCFVP |
| 50 mM Met | CCFVP | CCFVP | CCFVP | CCFVP | CCFVP | CCFVP | CCFVP | CCFVP | CCFVP |

CCFVP: Clear, colorless, free of visible particles

RP-HPLC data for rhPTH formulated with different methionine concentrations during the storage stability period shows a significant reduction in peptide oxidation when methionine was included as a part of the formulation. However, within assay variability, no significant differences in the oxidation of Met8 and Met18 peaks, or the percent main peak were observed among the different methionine concentrations studied.

Optimization of Poloxamer-188 Concentration

Agitation studies were used to optimize Poloxamer-188 concentration. Tables 20-22 show the visual appearance results for the samples with different concentrations of Poloxamer-188 (formulated with different methionine content—Table 17) in 2R vials that underwent horizontal orbital agitation at 220 rpm under ambient conditions.

TABLE 20
Visual appearance results for rhPTH samples formulated with different concentrations of
Poloxamer-188 (P-188) and 10 mM methionine at pH 5.5 with 20 mM acetate buffer and
0.3% m-cresol upon orbital agitation at 220 rpm under ambient conditions (n = 3)

| P-188 (%) | T0 | 8 h | 14 h | 24 h | 40 h | 48 h | 58 h |
|---|---|---|---|---|---|---|---|
| 0 | CCFVP | CCFVP | >SOP | >SOP | | | |
| (Control) | | | | | | | |
| 0.1 | CCFVP | CCFVP | CCFVP | CCFVP | CCFVP | Between RS I and RS II | SOP |
| | | | | | | (fine particles) | |
| 0.2 | CCFVP | CCFVP | CCFVP | CCFVP | CCFVP | CCFVP | CCFVP |
| 0.3 | CCFVP | CCFVP | CCFVP | CCFVP | CCFVP | CCFVP | RS II |
| | | | | | | | (fine particles) |
| 0.5 | CCFVP | CCFVP | CCFVP | CCFVP | CCFVP | Between RS I and RS II | >SOP |
| | | | | | | | (particles) |

CCFVP: Clear, colorless, free of visible particles; RS: Reference Suspension; SOP: Standard of Opalescence

TABLE 21
Visual appearance results for rhPTH samples formulated with different concentrations of
Poloxamer-188 (P-188) and 25 mM methionine at pH 5.5 with 20 mM acetate buffer and
0.3% m-cresol upon orbital agitation at 220 rpm under ambient conditions (n = 3)

| P-188 (%) | T0 | 8 h | 14 h | 24 h | 40 h | 48 h | 58 h |
|---|---|---|---|---|---|---|---|
| 0 | CCFVP | CCFVP | >SOP | >SOP | | | |
| (Control) | | | | | | | |
| 0.1 | CCFVP | CCFVP | CCFVP | CCFVP | CCFVP | CCFVP | CCFVP |
| 0.2 | CCFVP | CCFVP | CCFVP | CCFVP | CCFVP | Between RS II and RS III | >SOP |
| | | | | | | (fine particles) | (particles) |
| 0.3 | CCFVP | CCFVP | CCFVP | CCFVP | CCFVP | Between RS II and RS III | >SOP |
| | | | | | | (fine particles) | (particles) |
| 0.5 | CCFVP | CCFVP | CCFVP | CCFVP | CCFVP | Between W and RS I | >SOP |
| | | | | | | | (particles) |

CCFVP: Clear, colorless, free of visible particles; RS: Reference Suspension; W: Water like appearance; SOP: Standard of Opalescence

TABLE 22
Visual appearance results for rhPTH samples formulated with different concentrations of
Poloxamer-188 (P-188) and 50 mM methionine at pH 5.5 with 20 mM acetate buffer and
0.3% m-cresol upon orbital agitation at 220 rpm under ambient conditions (n = 3)

| P-188% | T0 | 8 h | 14 h | 24 h | 40 h | 48 h | 58 h |
|---|---|---|---|---|---|---|---|
| 0 (Control) | CCFVP | CCFVP | RS I (particles) | >SOP | | | |
| 0.3 | CCFVP | CCFVP | CCFVP | CCFVP | CCFVP | RS I | CCFVP |
| 0.5 | CCFVP | CCFVP | CCFVP | CCFVP | CCFVP | RS I | CCFVP |
| 0.75 | CCFVP | CCFVP | CCFVP | CCFVP | CCFVP | RS I | RS I |
| 1.0 | CCFVP | CCFVP | CCFVP | CCFVP | CCFVP | >SOP | >SOP |
| 0% P-188 siliconized | CCFVP | CCFVP | RS I | >SOP | >SOP | >SOP | >SOP |
| cartiridges | | | (Few particles) | | | | |
| 0.3% P-188 siliconized | CCFVP | CCFVP | CCFVP | CCFVP | CCFVP | CCFVP | CCFVP |
| cartridges | | | | | | | |

CCFVP: Clear, colorless, free of visible particles; RS: Reference Suspension; SOP: Standard of Opalescence

Selection of Stabilizers/Tonicity Agents

Sodium chloride (NaCl), sucrose, glycerol, and mannitol were selected as suitable excipients during the rhPTH excipient screening studies (Example 8). The concentrations to be used in future formulations were selected based on the osmolality target of 250-350 mOsm/kg.

All samples had clear to minimal opalescence over the studied duration without any visible particles present (Table 23).

TABLE 23
Visual appearance results for rhPTH stability samples formulated with different stabilizers/tonicity
agents in pH 5.5, 20 mM acetate buffer with 25 mM methionine, 0.3% Poloxamer-188, and 0.3%
m-cresol upon storage under different temperature conditions

| | | 40° C. | | | 25° C. | | | 5° C. | |
|---|---|---|---|---|---|---|---|---|---|
| Formulation | T0 | 1 m | 3 m | 6 m | 1 m | 3 m | 6 m | 3 m | 6 m |
| None | W amd RS I | W and RS I | W and RS I | CCFVP | CCFVP | W and RS I | CCFVP | W and RS I | CCFVP |
| (Control) | (no particles) | (no particles) | (no particles) | | | (no particles) | | (no particles) | |
| 130 mM | W amd RS I | W amd RS I | W amd RS I | CCFVP | W amd RS I | W amd RS I | CCFVP | W amd RS I | CCFVP |
| NaCl | (no particles) | (no particles) | (no particles) | | (no particles) | (no particles) | | (no particles) | |
| 8.5% w/v | W amd RS I | W amd RS I | W amd RS I | CCFVP | W amd RS I | CCFVP | CCFVP | W amd RS I | CCFVP |
| Sucrose | (no particles) | (no particles) | (no particles) | | (no particles) | | | (no particles) | |
| 2.3% v/v | W amd RS I | W amd RS I | W amd RS I | CCFVP | CCFVP | CCFVP | CCFVP | W amd RS I | CCFVP |
| Glycerol | (no particles) | (no particles) | (no particles) | | | | | (no particles) | |
| 250 mM | W amd RS I | W amd RS I | W amd RS I | CCFVP | CCFVP | CCFVP | CCFVP | W amd RS I | CCFVP |
| Mannitol | (no particles) | (no particles) | (no particles) | | | | | (no particles) | |

CCFVP: Clear, colorless, free of visible particles; RS: Reference Suspension; W: Water-like appearance; SOP: Standard of Opalescence

RP-HPLC data for rhPTH formulated in 20 mM acetate buffer with 25 mM methionine, 0.3% P-188, and 0.3% m-cresol with different stabilizers/tonicity agents upon storage at 40, 25, and 5° C. indicates no significant changes in oxidized Met8 and Met18 levels between the different excipients used at any of the incubation temperatures. The same trend was observed at 5° C. storage. The rate of formation of IsoAsp33 was similar for all studied excipients, except for NaCl, which contained a significantly lesser amount of IsoAsp33. The lower levels of IsoAsp33 along with a significantly reduced formation of unidentified tailing peak in NaCl formulation also resulted in the highest main peak recovery with NaCl when compared to other excipients.

Agitation studies: Formulations with different stabilizers (Table 18) were exposed to orbital agitation in 2R vials and siliconized cartridges at 220 rpm under ambient conditions. Table 24 lists the visual appearance results from one exemplary study days with multiple replicates in 2R vials. In some cases, the three replicates of individual vial did not show the same appearance profile during agitation; the worst of the visual appearance observations is reported.

All these formulations stayed clear and free of visible particles at the end of 72 hours when agitated horizontally in siliconized cartridges at 220 rpm under ambient conditions.

TABLE 24
Visual appearance results for rhPTH samples formulated with different stabilizers/tonicity agents
at pH 5.5 with 20 mM acetate buffer, 25 mM methionine, 0.3% Poloxamer-188, and 0.3% m-cresol
upon orbital agitation at 220 rpm under ambient conditions in 2R vials

| Formulation | T0 | 12 h | 24 h | 36 h | 48 h | 60 h | 72 h | 120 h |
|---|---|---|---|---|---|---|---|---|
| Control | Clear and free of visible particles (Between W and RSI) | | | | | | | >SOP |
| | | | | | | | | (fine particles) |
| 130 mM NaCl | Clear and free of visible particles | | RS II | >SOP | >SOP | >SOP | >SOP | >SOP |
| | (Between W and RSI) | | (fine particles) | (fine particles) | (fine particles) | (fine particles) | (fine particles) | (fine particles) |
| 8.5% Sucrose | Clear and fine of visible particles (Between W and RSI) | | | | | | | Between RS II |
| | | | | | | | | and RS III (haze) |
| 2.3% Glycerol | CCFVP | CCFVP | CCFVP | CCFVP | CCFVP | CCFVP | Clear and free | RS III |
| | | | | | | | of visible | (fine particles) |
| | | | | | | | particles | |
| | | | | | | | (Between | |
| | | | | | | | W and RSI) | |

| | | | |
|---|---|---|---|
| 250 mM | Cear and free of visible particles (Between W and RSI) | RS IV | >SOP (haze) |
| Mannitol | | (fine particles) | |

CCFVP: Clear, colorless, free of visible particles; W: Water-like appearance; RS: Reference Suspension; SOP: Standard of Opalescence

Amongst the tested formulations, control samples and samples containing sucrose and glycerol showed the best visual appearance profile upon agitation.

In conclusion, no differences between the oxidation profile of rhPTH were observed between 50 mM, 25 mM and 10 mM methionine containing formulations upon thermal stress. Visible particle formation in rhPTH solution upon agitation was not observed to be dependent on the concentration of Poloxamer-188. Stabilizers/tonicity agents, in the form of NaCl, sucrose, and glycerol, were selected from a combination of chemical and physical changes in the molecule observed upon thermal and agitation stresses performed during the preliminary excipient screen (see Example 7). The concentration of stabilizers/tonicity agents were selected to achieve isotonic solutions. Based on the overall data from thermal and agitation stresses, three top formulation matrices targeting a liquid dosage form were identified:

a) pH 5.5, 20 mM Acetate Buffer, 10 mM Methionine, 0.3% w/v Poloxamer-188, 130 mM Sodium Chloride, 0.3% w/v m-cresol

b) pH 5.5, 20 mM Acetate Buffer, 10 mM Methionine, 0.3% w/v Poloxamer-188, 8.5% w/v Sucrose, 0.3% w/v m-cresol

c) pH 5.5, 20 mM Acetate Buffer, 10 mM Methionine, 0.3% w/v Poloxamer-188, 2.3% v/v Glycerol, 0.3% w/v m-cresol

The target concentrations of rhPTH in these formulations range between 0.35 mg/mL to 1.4 mg/mL.

### Example 10: Development of a Multi-Dose Lyophilized rhPTH Drug Product for Subcutaneous Delivery

Reformulation studies were performed to understand the effect of pH, buffers, surfactants, stabilizers/bulking agents on the chemical and physical stability of lyophilized rhPTH (1-84). Chemical stability of rhPTH (1-84) is greatly affected by solution pH, with optimal stability observed in the pH range of 5.0 to 6.5. Lower pH (4.0-4.5) significantly increased fragmentation of rhPTH (1-84) while improving stability against shaking-induced particulate formation. At higher pH (above 6.5), reconstituted lyophilized formulations of rhPTH (1-84) were increasingly prone to particulate formation.

At the optimal solution pH of 5.5, formulations containing L-histidine and phosphate buffer showed a significant improvement against visible particulate formation, upon shaking in 2R vials and siliconized cartridges, when compared to citrate buffer-containing formulations. The addition of poloxamer-188 to L-histidine formulation at pH 5.5 further improved rhPTH(1-84) stability against shaking-induced particulate formation.

Succinate buffer at pH 4.0 to 4.3 was also identified as another buffer candidate as it seemed to provide complete protection against shaking-induced particulate formation, although providing inferior chemical stability when compared to other buffers at pH 5.5.

Overall, the results from these screening studies have helped identify three leading lyophilized rhPTH (1-84) formulation candidates for further evaluation with the current commercial dual-chamber cartridges. Selection of these formulations was mainly based on the results from real-time, accelerated, and stressed storage stability studies as well as shaking-induced stress studies following reconstitution with 0.3% (v/v) m-cresol solution in water. The three lyophilized formulation candidates, which will require reconstitution with WFI containing 0.3% (w/v) m-cresol prior to use, consist of the following:

1. 1 mg/mL rhPTH (1-84) in 20 mM L-histidine at pH 5.5 with 4% (w/v) mannitol and 2% (w/v) sucrose

2. 1 mg/mL rhPTH (1-84) in 20 mM L-histidine at pH 5.5 with 4% (w/v) mannitol, 2% (w/v) sucrose, and 0.3% (w/v) poloxamer-188

3. 1 mg/mL rhPTH (1-84) in 20 mM succinate at pH 4.3 with 3% (w/v) mannitol and 3% (w/v) sucrose

For monitoring the chemical stability of rhPTH (1-84), reversed phase-high-performance liquid chromatography (RP-HPLC) was used to quantitate impurities associated with oxidation, deamidation, fragmentations, and others degradation pathways. Size-exclusion chromatography (SEC) was used to quantitate any high and low molecular species in addition to the main rhPTH (1-84) molecule.

For evaluation of the physical stress on rhPTH (1-84), agitation by orbital shaking was employed and visual appearance was used to assess the results. All formulations were reconstituted with 0.3% (v/v) m-cresol solution in water and subjected to shaking at room temperature in a horizontal position using an orbital shaker set at 220 rpm.

The water content measured using Karl Fisher is summarized in Table 25. All formulations had less than 2% water content, except the formulation containing 100 mM sodium chloride and 5% sucrose. Water content of less than 2% should pose no stability issue with rhPTH (1-84) as it is significantly lower than the commercial drug product water content specification.

TABLE 25
Summary of Water Content by Karl Fisher

| Formulation Description | Water Content |
|---|---|
| 0.5 mg/mL rhPTH (1-84), | 1.0% |
| 50 mM NaCl, 5% Sucrose | |
| 0.5 mg/mL rhPTH (1-84), | 1.0% |
| 30 mM NaCl, 5% Sucrose | |
| 0.5 mg/mL rhPTH (1-84), | 1.5% |
| 75 mM NaCl, 5% Sucrose | |
| 0.5 mg/mL rhPTH (1-84), | 3.4% |
| 100 mM NaCl, 5% Sucrose | |
| 0.5 mg/mL rhPTH (1-84), | 1.6% |
| 100 mM NaCl, 8% Sucrose | |
| 0.5 mg/mL rhPTH (1-84), | 0.7% |
| 30 mM NaCl, 8% Sucrose | |
| 0.5 mg/mL rhPTH (1-84), | 0.9% |
| 50 mM NaCl, 8% Sucrose | |
| 0.5 mg/mL rhPTH (1-84), | 1.5% |
| 75 mM NaCl, 8% Sucrose | |

Based on the collective results, a formulation consisting of 30 mM sodium chloride and 5% (w/v) sucrose with a Tg′ of around −39° C. was chosen for initial lyophilization work as it yielded a decent cake appearance with low moisture content.

Reformulation studies of lyophilized rhPTH (1-84) confirmed the optimal pH range of 5.0-6.0 which provided the least chemical degradation for rhPTH (1-84). Although rhPTH (1-84) degraded rather fast at elevated temperatures of 25° C. and 40° C. at lower pH condition, studies also identified that rhPTH (1-84) formulation at pH of 4.0-4.3 may still be possible as it maintained chemical stability at storage condition of 5° C. for up to 6 months and significantly reduced the shaking-induced particulate formation. As a result of extensive lyophilized formulation screening studies, coupled with concurrent liquid formulation development of rhPTH (1-84), three leading lyophilized formulations of rhPTH (1-84) were selected for evaluation with the current commercial siliconized dual-chamber cartridges. The chosen formulations were based on 3 months stability at accelerated and stressed conditions of 25° C. and 40° C., respectively, and their effects on rhPTH (1-84) stability against shaking-induced particulate formation following reconstitution with 0.3% (v/v) m-cresol in water. The three selected formulations are:

Additional optimization studies indicated that the addition of 10 mM methionine significantly improved the stability of rhPTH (1-84) against oxidation of Met8 and Met18 residues in the Formulation 2 and against aggregation in Formulation 3 above.

As various changes can be made in the above-described subject matter without departing from the scope and spirit of the present invention, it is intended that all subject matter contained in the above description, or defined in the appended claims, be interpreted as descriptive and illustrative of the present invention. Many modifications and variations of the present invention are possible in light of the above teachings. Accordingly, the present description is intended to embrace all such alternatives, modifications, and variances which fall within the scope of the appended claims.

All patents, applications, publications, test methods, literature, and other materials cited herein are hereby incorporated by reference in their entirety as if physically present in this specification.

1. A pharmaceutical formulation comprising:
(a) a therapeutically effective amount of recombinant human parathyroid hormone (rhPTH(1-84));
(b) a surfactant;
(c) a tonicity agent;
(d) an antioxidant;
(e) a preservative;
(f) a physiologically acceptable buffer, and
(g) water,
wherein said pharmaceutical formulation is formulated as a liquid for injection, and wherein the formulation is physically stable and remains clear, colorless, and free of visible particles for at least 48 hours.

2. The pharmaceutical formulation of claim 1, wherein the formulation is physically stable for at least 72 hours.

3. The pharmaceutical formulation of claim 1, wherein the formulation is physically stable for at least 96 hours.

4. The pharmaceutical formulation of claim 1, wherein the formulation is physically stable for at least 7 days.

5. The pharmaceutical formulation of claim 1, wherein the formulation is physically stable for at least 14 days.

6. The pharmaceutical formulation of claim 1, wherein the formulation is physically stable for at least 21 days.

7. The pharmaceutical formulation of claim 1, wherein the surfactant is selected from Poloxamer-188 and polyethylene glycol, and combinations thereof.

8. The pharmaceutical formulation of claim 1, wherein the tonicity agent is selected from sodium chloride, sucrose, and glycerol, and combinations thereof.

9. The pharmaceutical formulation of claim 1, wherein the preservative is m-cresol, phenol, benzyl alcohol, sodium benzoate, propyl paraben, or combinations thereof.

10. The pharmaceutical formulation of claim 1, wherein the physiologically acceptable buffer is acetate buffer, phosphate buffer, L-Histidine buffer, or succinate buffer.

11. The pharmaceutical formulation of claim 1, further comprising an antioxidant.

12. The pharmaceutical formulation of claim 11, wherein the antioxidant is methionine, N-Acetyl-methionine, thiosulfate, N-Acetyl tryptophan, or combinations thereof.

13. The pharmaceutical formulation of claim 1 having a pH of about 4 to about 6.

14. The pharmaceutical formulation of claim 1 having a pH of about 5.5.

15. The pharmaceutical formulation of claim 1, wherein the formulation is in a unit-dose vial, a multi-dose vial, a cartridge, a pre-filled syringe, or an injection pen.

16. A pharmaceutical formulation comprising:
(a) about 0.2 to about 2.0 mg/mL recombinant human parathyroid hormone (rhPTH(1-84));
(b) about 0.03% to about 3.0% w/v surfactant;
(c) about 0.2% to about 20% w/v tonicity agent;
(d) about 0.015% to about 1.50% w/v antioxidant;
(e) about 0.03% to about 3% preservative;
(f) about 5 mM to about 50 mM physiologically acceptable buffer, and
(g) water,
wherein said pharmaceutical formulation is formulated as a liquid for injection, and wherein the formulation is physically stable and remains clear, colorless, and free of visible particles for at least 48 hours.

17. The pharmaceutical formulation of claim 16, wherein the formulation is physically stable for at least 72 hours.

18. The pharmaceutical formulation of claim 16, wherein the formulation is physically stable for at least 96 hours.

19. The pharmaceutical formulation of claim 16, wherein the formulation is physically stable for at least 7 days.

20. The pharmaceutical formulation of claim 16, wherein the formulation is physically stable for at least 14 days.

21. The pharmaceutical formulation of claim 16, wherein the formulation is physically stable for at least 21 days.

22. A pharmaceutical formulation comprising:
(a) a therapeutically effective amount of recombinant human parathyroid hormone (rhPTH(1-84));
(b) a bulking agent;
(c) a cryoprotectant, and
(d) a pharmaceutically acceptable buffer,
wherein said pharmaceutical formulation is formulated as a lyophilized powder to be reconstituted prior to injection, and wherein the formulation is physically stable and remains clear, colorless, and free of visible particles for at least 48 hours after reconstitution.

23. The pharmaceutical formulation of claim 22, wherein the formulation is physically stable for at least 72 hours.

24. The pharmaceutical formulation of claim 22, wherein the formulation is physically stable for at least 96 hours.

25. The pharmaceutical formulation of claim 22, wherein the formulation is physically stable for at least 7 days.

26. The pharmaceutical formulation of claim 22, wherein the formulation is physically stable for at least 14 days.

27. The pharmaceutical formulation of claim 22, wherein the formulation is physically stable for at least 21 days.

28. The pharmaceutical formulation of claim 22, wherein the bulking agent is mannitol.

29. The pharmaceutical formulation of claim 22, wherein the cryoprotectant is sucrose.

30. The pharmaceutical formulation of claim 22, wherein the pharmaceutically acceptable buffer is acetate buffer, phosphate buffer, L-Histidine buffer, or succinate buffer.

31. The pharmaceutical formulation of claim 22, wherein the pharmaceutically acceptable buffer is L-Histidine buffer.

32. The pharmaceutical formulation of claim 31 having a pH of about 5.5.

33. The pharmaceutical formulation of claim 22, wherein the pharmaceutically acceptable buffer is succinate buffer.

34. The pharmaceutical formulation of claim 33 having a pH of between about 4 and about 4.5.

35. The pharmaceutical formulation of claim 22, further comprising an antioxidant and/or a surfactant.

36. The pharmaceutical formulation of claim 35, wherein the antioxidant is methionine and the surfactant is Poloxamer-188.

37. A pharmaceutical formulation comprising:
(a) about 0.02 to about 2.0 mg/mL recombinant human parathyroid hormone (rhPTH(1-84));
(b) about 0.3% to about 30% w/v bulking agent;
(c) about 0.2% to about 20% w/v cryoprotectant, and
(d) about 5 mM to about 50 mM pharmaceutically acceptable buffer,
wherein said pharmaceutical formulation is formulated as a lyophilized powder to be reconstituted prior to injection, and wherein the formulation is physically stable and remains clear, colorless, and free of visible particles for at least 48 hours after reconstitution.

38. The pharmaceutical formulation of claim 37, wherein the formulation is physically stable for at least 72 hours.

39. The pharmaceutical formulation of claim 37, wherein the formulation is physically stable for at least 96 hours.

40. The pharmaceutical formulation of claim 37, wherein the formulation is physically stable for at least 7 days.

41. The pharmaceutical formulation of claim 37, wherein the formulation is physically stable for at least 14 days.

42. The pharmaceutical formulation of claim 37, wherein the formulation is physically stable for at least 21 days.

43. A kit for formulating an injectable solution of rhPTH(1-84) comprising a first container comprising the pharmaceutical formulation of claim 22, a second container comprising sterile water for reconstituting said pharmaceutical formulation, and a sheet instructing preparation of a reconstituted formulation therefrom.

44. The kit of claim 43, further comprising a device for injection of the reconstituted rhPTH(1-84) solution.

45. A method of administering a therapeutically effective amount of rhPTH(1-84) to a subject in need thereof, comprising subcutaneously, intravenously, or intramuscularly injecting the pharmaceutical formulation of claim 1 into the subject.

46. The method of claim 45, wherein the injecting is performed with a syringe, an auto-injector, an injection pen, or a combination thereof.

47. A method of administering a therapeutically effective amount of rhPTH(1-84) to a subject in need thereof, comprising
(i) reconstituting the pharmaceutical formulation of claim 22 with sterile water, and
(ii) subcutaneously, intravenously, or intramuscularly injecting the reconstituted formulation into the subject.

48. The method of claim 47, wherein the injecting is performed with a syringe, an auto-injector, an injection pen, or a combination thereof.
